(19) United States
(12) Patent Application Publication
(10) Pub. No.: US 2026/0051394 A1
(43) Pub. Date: Feb. 19, 2026

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: TOSHIHIRO USUDA, Tokyo (JP)

(73) Assignee: FUJIFILM Corporation, Tokyo (JP)

(21) Appl. No.: 19/298,206

(22) Filed: Aug. 13, 2025

(30) Foreign Application Priority Data
Aug. 15, 2024 (JP) 2024-135707

(51) Int. Cl. G16H 30/40; G06T 7/00; G06T 7/11

(52) U.S. Cl. CPC G16H 30/40; G06T 7/0012; G06T 7/11; G06T 2207/10068; G06T 2207/30096

(57) ABSTRACT

A medical support device includes a processor. A processor is configured to: acquire a medical image obtained by imaging a portion including a feature region; and perform first processing in accordance with a sharpness level of an inner region, which is a region inside an outer edge of the feature region included in the medical image, or second processing in accordance with the sharpness level, in which the first processing is processing of controlling image recognition processing that is executable on the medical image and that recognizes a feature of the feature region, and the second processing is processing of controlling output of information based on a processing result of the image recognition processing.

### CROSS-REFERENCE TO RELATED APPLICATIONS

This application claims priority under 35 USC 119 from Japanese Patent Application No. 2024-135707 filed on Aug. 15, 2024, the disclosure of which is incorporated by reference herein.

BACKGROUND

### 1. Technical Field

The present disclosure relates to a medical support device, an endoscope system, a medical support method, and a program.

### 2. Related Art

WO2023/144936A discloses an image determination device comprising an acquisition unit, a detection unit, a setting unit, a blurriness determination unit, and an image determination unit. In the image determination device disclosed in WO2023/144936A, the acquisition unit acquires an endoscopic image. The detection unit detects a lesion candidate from the endoscopic image, and outputs a lesion candidate image including the lesion candidate. The setting unit sets a lesion region corresponding to the lesion candidate and a correspondence region corresponding to the lesion region in the lesion candidate image. The blurriness determination unit determines blurriness of an image of the lesion region based on an image of the lesion region and an image of the correspondence region. Further, the blurriness determination unit determines the blurriness of the image of the lesion region based on power of a high-frequency component for each of the image of the lesion region and the image of the correspondence region. The image determination unit determines suitability of the lesion candidate image based on a determination result of the blurriness.

WO2019/142243A discloses an image diagnostic support system comprising an input unit, a specifying unit, and a determination unit. In the image diagnostic system disclosed in WO2019/142243A, the input unit receives an input of an image. The specifying unit specifies a specular reflection region and a non-specular reflection region in a region of interest in the image. The determination unit determines whether or not the region of interest is a non-suitable region that is not suitable for diagnosis based on an image processing result for at least one of the specular reflection region or the non-specular reflection region. In addition, the determination unit determines whether or not the region of interest is a non-suitable region accompanied by blurriness based on the image processing result for the non-specular reflection region.

The image diagnostic system disclosed in WO2019/142243A further comprises a blurriness amount calculation unit that calculates a blurriness amount of the non-specular reflection region. The blurriness amount calculation unit calculates the blurriness using an image before application of a Gaussian filter and an image after application of the Gaussian filter. The determination unit determines whether or not the region of interest is the non-suitable region accompanied by blurriness based on the blurriness amount calculated by the blurriness amount calculation unit.

### SUMMARY

One embodiment according to the present disclosure provides a medical support device, an endoscope system, a medical support method, and a program that can prevent information, in which a result of erroneous recognition of a feature of a feature region shown in a medical image by image recognition processing due to a low accuracy of a sharpness level of the feature region is reflected, from being provided to a user or the like.

A first aspect according to the present disclosure relates to a medical support device comprising: a processor configured to: acquire a medical image obtained by imaging a portion including a feature region; and perform first processing in accordance with a sharpness level of an inner region, which is a region inside an outer edge of the feature region included in the medical image, or second processing in accordance with the sharpness level, in which the first processing is processing of controlling image recognition processing that is executable on the medical image and that recognizes a feature of the feature region, and the second processing is processing of controlling output of information based on a processing result of the image recognition processing.

A second aspect according to the present disclosure relates to the medical support device according to the first aspect, in which the first processing includes processing of executing the image recognition processing in a case in which the sharpness level is equal to or greater than a first threshold value and not executing the image recognition processing in a case in which the sharpness level is less than a second threshold value that is equal to or less than the first threshold value.

A third aspect according to the present disclosure relates to the medical support device according to the first or second aspect, in which the second processing includes processing of outputting the information in a case in which the sharpness level is equal to or greater than a first threshold value and not outputting the information in a case in which the sharpness level is less than a second threshold value that is equal to or less than the first threshold value.

A fourth aspect according to the present disclosure relates to the medical support device according to any one of the first to third aspects, in which region recognition processing of recognizing the feature region is executed on the medical image, and the inner region is a region based on a segmentation mask obtained by executing the region recognition processing.

A fifth aspect according to the present disclosure relates to the medical support device according to the fourth aspect, in which a resolution of the segmentation mask is lower than a resolution of the medical image.

A sixth aspect according to the present disclosure relates to the medical support device according to any one of the first to third aspects, in which the inner region is a region within a second frame that is a frame obtained by narrowing a first frame, which is a frame surrounding the feature region, to an inner side of the feature region from the outer edge.

A seventh aspect according to the present disclosure relates to the medical support device according to the sixth aspect, in which region recognition processing of recognizing the feature region is executed on the medical image, a bounding box is used in the region recognition processing, and the first frame corresponds to the bounding box.

An eighth aspect according to the present disclosure relates to the medical support device according to any one of the first to seventh aspects, in which, in a case in which a part of the feature region included in the medical image deviates from the medical image, the inner region is a region excluding an edge of the medical image.

A ninth aspect according to the present disclosure relates to the medical support device according to any one of the first to eighth aspects, in which, in a case in which a halation portion exists in the feature region included in the medical image, the inner region is a region outside an edge of the halation portion.

A tenth aspect according to the present disclosure relates to the medical support device according to any one of the first to ninth aspects, in which the first processing and/or the second processing is executed in accordance with a plurality of the sharpness levels obtained from a plurality of the medical images arranged in time series.

An eleventh aspect according to the present disclosure relates to the medical support device according to any one of the first to tenth aspects, in which a weight determined based on a plurality of the sharpness levels is given to at least one processing result among a plurality of the processing results obtained by executing the image recognition processing on the plurality of medical images.

A twelfth aspect according to the present disclosure relates to the medical support device according to any one of the first to eleventh aspects, in which the output of the information includes display of the information on a screen.

A thirteenth aspect according to the present disclosure relates to the medical support device according to any one of the first to twelfth aspects, in which the feature is a medical feature of the feature region.

A fourteenth aspect according to the present disclosure relates to the medical support device according to any one of the first to the thirteenth aspects, in which the feature region is a lesion.

A fifteenth aspect according to the present disclosure relates to the medical support device according to any one of the first to fourteenth aspects, in which the medical image is an endoscopic image.

A sixteenth aspect according to the present disclosure relates to an endoscope system comprising: the medical support device according to any one of the first to fifteenth aspects; and an endoscope in which an image sensor that images the portion is mounted.

A seventeenth aspect according to the present disclosure relates to a medical support method comprising: acquiring a medical image obtained by imaging a portion including a feature region; and performing first processing in accordance with a sharpness level of an inner region, which is a region inside an outer edge of the feature region included in the medical image, or second processing in accordance with the sharpness level, in which the first processing is processing of controlling image recognition processing that is executable on the medical image and that recognizes a feature of the feature region, and the second processing is processing of controlling output of information based on a processing result of the image recognition processing.

An eighteenth aspect according to the present disclosure relates to a program causing a computer to execute medical support processing comprising: acquiring a medical image obtained by imaging a portion including a feature region; and performing first processing in accordance with a sharpness level of an inner region, which is a region inside an outer edge of the feature region included in the medical image, or second processing in accordance with the sharpness level, in which the first processing is processing of controlling image recognition processing that is executable on the medical image and that recognizes a feature of the feature region, and the second processing is processing of controlling output of information based on a processing result of the image recognition processing.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope system is used by a doctor;

FIG. 2 is a conceptual diagram showing an example of an overall configuration of the endoscope system;

FIG. 3 is a block diagram showing an example of a hardware configuration of an electrical system of the endoscope system;

FIG. 4 is a conceptual diagram showing an example of an aspect in which feature recognition processing is performed in accordance with a sharpness level of an image of interest including a sharp region and a non-sharp region;

FIG. 5 is a conceptual diagram showing an example of an aspect in which the feature recognition processing is performed in accordance with the sharpness level of the image of interest in which a lesion in which a halation portion is present is shown;

FIG. 6 is a conceptual diagram showing an example of an aspect in which the feature recognition processing is performed in accordance with the sharpness level of the image of interest in which an edge of a frame is shown together with the lesion;

FIG. 7 is a block diagram showing an example of main functions of a processor provided in a medical support device and an example of information stored in a storage;

FIG. 8 is a conceptual diagram showing an example of an aspect in which first region recognition processing is executed by a recognition unit and various types of information are displayed on a screen by a controller;

FIG. 9 is a conceptual diagram showing an example of an aspect in which second region recognition processing is executed by the recognition unit;

FIG. 10 is a conceptual diagram showing an example of an aspect in which the recognition unit reduces an outer shape of a segmentation mask and fits an outer edge of the segmentation mask to a processing target image;

FIG. 11 is a conceptual diagram showing an example of an aspect in which an inner region is extracted from the processing target image by the recognition unit and first control processing is executed in accordance with a sharpness level of the inner region;

FIG. 12 is a conceptual diagram showing an example of processing contents of the feature recognition processing executed by the recognition unit;

FIG. 13 is a conceptual diagram showing an example of an aspect in which second region recognition processing is executed on the processing target image in which the lesion in which the halation portion is present is shown;

FIG. 14 is a conceptual diagram showing an example of an aspect in which a contour of the segmentation mask in which a blank region in the segmentation mask obtained by executing the second region recognition processing on the processing target image in which the lesion in which the halation portion is present is shown is enlarged and the outer shape of the segmentation mask is reduced is fitted to the processing target image;

FIG. 15 is a conceptual diagram showing an example of an aspect in which the inner region is extracted from the processing target image to which the contour of the segmentation mask is fitted by the recognition unit, and the first control processing is executed in accordance with the sharpness level of the inner region;

FIG. 16 is a conceptual diagram showing an example of an aspect in which the feature recognition processing is executed on the inner region extracted from the processing target image by the recognition unit;

FIG. 17 is a conceptual diagram showing an example of an aspect in which the second region recognition processing is executed on the processing target image in which the edge of the frame is shown together with the lesion in which the halation portion is present;

FIG. 18 is a conceptual diagram showing an example of an aspect in which the contour of the segmentation mask in which the blank region in the segmentation mask obtained by executing the second region recognition processing on the processing target image in which the edge of the frame is shown together with the lesion in which the halation portion is present is enlarged and the outer shape of the segmentation mask is reduced is fitted to the processing target image;

FIG. 19 is a conceptual diagram showing an example of an aspect in which the inner region is extracted from the processing target image to which the contour of the segmentation mask is fitted by the recognition unit, and the first control processing is executed in accordance with the sharpness level of the inner region;

FIG. 20 is a conceptual diagram showing an example of an aspect in which the feature recognition processing is executed on the inner region extracted from the processing target image by the recognition unit;

FIG. 21 is a conceptual diagram showing an example of an aspect in which the controller displays processing result information on the screen;

FIG. 22 is a flowchart showing an example of a flow of medical support processing;

FIG. 23 is a conceptual diagram showing a modification example of an aspect in which the inner region is extracted from the processing target image;

FIG. 24 is a conceptual diagram showing an example of an aspect in which the second control processing is executed by the controller;

FIG. 25 is a flowchart showing a modification example of the flow of the medical support processing;

FIG. 26 is a conceptual diagram showing an example of an aspect in which the sharpness level obtained based on a latest frame is updated based on a plurality of sharpness levels and a plurality of malignancy degrees obtained based on a plurality of frames arranged in time series; and

FIG. 27 is a conceptual diagram showing an example of a series of processing in which a processor included in a computer issues a processing execution instruction to an external device via a network, the external device executes processing in response to the processing execution instruction, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings. The present disclosure is also applicable to a program and a computer program product.

The terms used in the following description will be described first.

CPU is an abbreviation for “central processing unit”. GPU is an abbreviation for “graphics processing unit”. GPGPU is an abbreviation for “general-purpose computing on graphics processing units”. APU is an abbreviation for “accelerated processing unit”. TPU is an abbreviation for “tensor processing unit”. RAM is an abbreviation for “random-access memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. EL is an abbreviation for “electro-luminescence”. CMOS is an abbreviation for “complementary metal oxide semiconductor”. CCD is an abbreviation for “charge-coupled device”. AI is an abbreviation for “artificial intelligence”. WLI is an abbreviation for “white light imaging”. BLI is an abbreviation for “blue light imaging”. LCI is an abbreviation for “linked color imaging”. NBI is an abbreviation for “narrow band imaging”. CT is an abbreviation for “computed tomography”. MRI is an abbreviation for “magnetic resonance imaging”. Mask R-CNN is an abbreviation for “mask regional convolutional neural network”. I/F is an abbreviation for “interface”. SSL is an abbreviation for “sessile serrated lesion”. LAN is an abbreviation for “local area network”. WAN is an abbreviation for “wide area network”. 5G is an abbreviation for “5th generation mobile communication system”.

Hereinafter, a processor with a reference numeral (hereinafter, simply referred to as a “processor”) may be one computing device or may be a combination of a plurality of computing devices. Furthermore, the processor may be one type of computing device or may be a combination of a plurality of types of computing devices. Examples of the computing device include a CPU, a GPU, a GPGPU, an APU, and a TPU.

In the following description, a memory with a reference numeral is a memory, such as a RAM, that temporarily stores information, and is used by the processor as a work memory.

In the following description, a storage with a reference numeral is one or a plurality of non-volatile storage devices that store various programs, various parameters, and the like. Examples of the non-volatile storage device include a flash memory, a magnetic disk, and a magnetic tape. Further, examples of the storage include a cloud storage.

In the embodiment described below, an external I/F with a reference numeral controls the transmission and reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), and Bluetooth (registered trademark).

In the embodiment described below, the expression “A and/or B” is synonymous with the expression “at least one of A or B”. That is, the expression “A and/or B” may mean only A, may mean only B, or may mean a combination of A and B. In addition, in the present specification, the same concept as the expression “A and/or B” is applied to a case in which the connection of three or more matters is expressed by “and/or”.

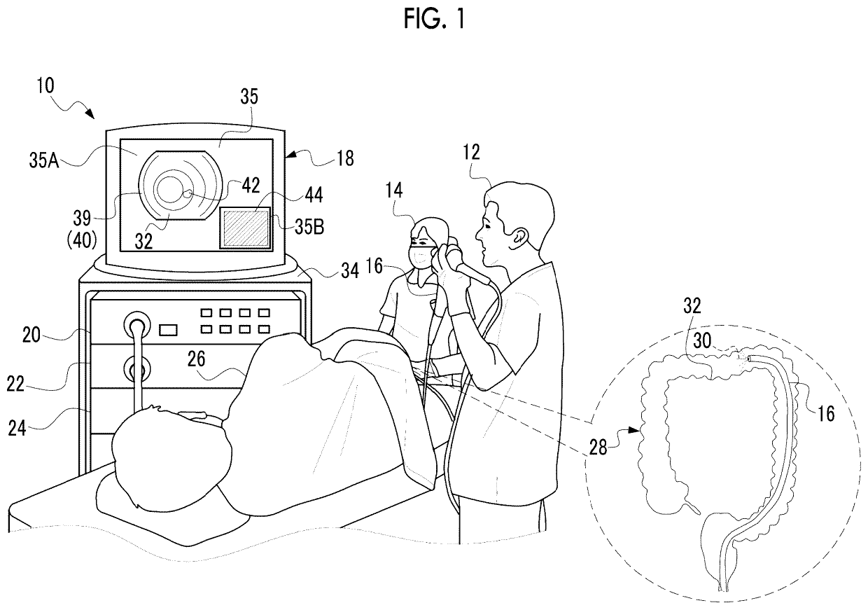

FIG. 1 is a conceptual diagram showing an example of an aspect in which an endoscope system 10 is used. As shown in FIG. 1, an endoscope system 10 is used by a doctor 12 in an endoscopy and the like. A staff member 14 such as a nurse assists with the endoscopy.

The endoscope system 10 comprises an endoscope 16, a display device 18, a control device 20, a light source device 22, and a medical support device 24. The endoscope system 10 in the present embodiment is an example of an “endoscope system” according to the present disclosure. In addition, the medical support device 24 in the present embodiment is an example of a “medical support device” according to the present disclosure.

The endoscope system 10 is a modality for a doctor 12 to perform medical care on a large intestine 28 included inside a body of a subject 26 (for example, a patient) using the endoscope 16. The large intestine 28 in the present embodiment is an example of a “portion” according to the present disclosure. Here, although the lower endoscopy has been described for the purpose of medical care for the large intestine 28, this is merely an example, and the present disclosure can also be applied to endoscopy for a luminal organ other than the large intestine 28 (for example, a luminal organ such as an esophagus, a stomach, a duodenum, or a trachea) such as an upper endoscopy.

The endoscope 16 is used by the doctor 12, and is inserted into the large intestine 28 of the subject 26. The endoscope system 10 causes the endoscope 16 inserted into the large intestine 28 to image the inside of the large intestine 28, and performs various medical treatments on the large intestine 28 as necessary.

The endoscope 16 irradiates the inside of the large intestine 28 with light 30. The endoscope 16 irradiates a region including an intestinal wall 32 with the light 30 to capture subject light that is reflected light obtained by being reflected in the region.

The display device 18, the control device 20, the light source device 22, and the medical support device 24 are installed in a wagon 34. The wagon 34 is provided with a plurality of tables along an up-down direction, and the medical support device 24, the light source device 22, and the control device 20 are installed from a lower table to an upper table. Moreover, the display device 18 is installed on an uppermost table in the wagon 34.

The control device 20 controls the entire endoscope system 10. For example, the control device 20 is used for the endoscope 16, the display device 18, the light source device 22, the medical support device 24, and the like, and the endoscope 16, the display device 18, the light source device 22, the medical support device 24, and the like are controlled by the control device 20.

The light source device 22 generates the light 30 and supplies the generated light 30 to the endoscope 16, under the control of the control device 20. A light guide (not shown) is built in the endoscope 16, and the light 30 supplied from the light source device 22 is emitted from a distal end portion of the endoscope 16 via the light guide.

The medical support device 24 performs various types of image processing on the image obtained by performing imaging with the endoscope 16, under the control of the control device 20.

The display device 18 displays various types of information including the image. Examples of the display device 18 include a liquid-crystal display and an EL display. Furthermore, a tablet terminal equipped with a display may be used instead of the display device 18 or together with the display device 18.

The display device 18 has a screen 35. A plurality of display regions are included in the screen 35. The plurality of display regions are arranged in the screen 35. In the example shown in FIG. 1, a first display region 35A and a second display region 35B are shown as examples of the plurality of display regions. A size of the first display region 35A is larger than a size of the second display region 35B. The first display region 35A is used as a main display region, and the second display region 35B is used as a sub-display region. A size relationship between the first display region 35A and the second display region 35B is not limited to this, and need only be a size relationship that can be included within the screen 35.

A video image 39 is displayed in the first display region 35A. The video image 39 is obtained by imaging the inside of the large intestine 28 of the subject 26 with the endoscope 16. In the example shown in FIG. 1, a video image in which the intestinal wall 32 is shown is shown as an example of the video image 39.

The intestinal wall 32 (that is, the intestinal wall 32 included as an image in the video image 39) shown in the video image 39 includes a lesion 42 (for example, in the example shown in FIG. 1, one lesion 42) as a region of interest (that is, an observation target region) that is gazed at by the doctor 12. The doctor 12 can visually recognize an aspect of the intestinal wall 32 including the lesion 42 through the video image 39. The lesion 42 in the present embodiment is an example of a “feature region” and a “lesion” according to the present disclosure.

There are various types of lesions 42, and examples of the types of the lesion 42 include adenoma, inflammatory polyp, hyperplastic polyp, serrated polyp, lymphoid polyp, adenocarcinoma, mucinous adenocarcinoma, sclerosing adenocarcinoma, squamous cell carcinoma, and malignant lymphoma. In addition, the types shown here are types assumed in advance as the types of the lesion 42 in a case in which the endoscopy is performed on the large intestine 28, and the types of the lesion 42 may be different depending on the organ on which the endoscopy is performed.

In the present embodiment, for convenience of description, the form example is described in which one lesion 42 is shown in the video image 39, but the present disclosure is not limited thereto, and the present disclosure is applicable even in a case in which a plurality of lesions 42 are shown in the video image 39.

In the present embodiment, the lesion 42 is shown, but this is merely an example, and the region of interest (that is, the observation target region) gazed at by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The video image 39 displayed in the first display region 35A is a video image including a plurality of frames 40 arranged in time series. That is, the plurality of frames 40 arranged in time series are displayed in the first display region 35A at a predetermined frame rate (for example, several tens of frames/second). Examples of the predetermined frame rate include 15 frames/second, 30 frames/second, and 60 frames/second. The frame 40 in the present embodiment is an example of a “medical image” and an “endoscopic image” according to the present disclosure.

Examples of the video image displayed in the first display region 35A include a video image in a live view mode. The live view mode is merely an example, and the video image may be a video image, such as a video image in a post view mode, that is temporarily stored in a memory or the like and then displayed. In addition, each frame included in a recording video image stored in a memory or the like may be reproduced and displayed on the screen 35 (for example, the first display region 35A) as the video image 39.

In the screen 35, the second display region 35B is adjacent to the first display region 35A, and is displayed at the lower right in the screen 35 in front view. The display position of the second display region 35B may be any position as long as the display position is located within the screen 35 of the display device 18, but it is preferable that the second display region 35B is displayed at a position comparable to the video image 39.

Auxiliary information 44 for assisting the doctor 12 in a medical determination or the like is displayed in the second display region 35B. The auxiliary information 44 is information to be referred to by the doctor 12. Examples of the auxiliary information 44 include various types of information on the subject 26 in which the endoscope 16 is inserted and various types of information obtained by executing medical support processing, which will be described later.

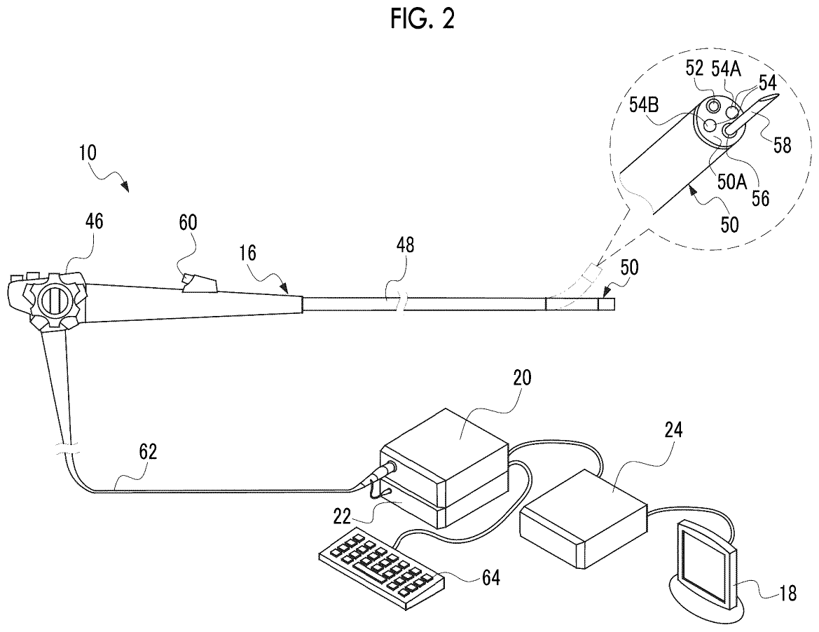

FIG. 2 is a conceptual diagram showing an example of an overall configuration of the endoscope system 10. As shown in FIG. 2, the endoscope 16 comprises an operating part 46 and an insertion part 48. The insertion part 48 is partially curved by the operation of the operating part 46. The insertion part 48 is inserted into the large intestine 28 while being curved along the shape of the large intestine 28 (see FIG. 1) in accordance with the operation of the operating part 46 performed by the doctor 12 (see FIG. 1).

A camera 52, an illumination device 54, and a treatment tool opening 56 are provided at a distal end portion 50 of the insertion part 48. The camera 52 and the illumination device 54 are provided at the distal end portion 50. A distal end surface 50A of the distal end portion 50 is provided with an objective lens of the camera 52.

The camera 52 is mounted in the endoscope 16, is inserted into a body cavity of the subject 26, and images the observation target region to generate the frame 40. In the present embodiment, the camera 52 generates the video image 39 including the plurality of frames 40 arranged in time series by imaging the inside of the body (for example, the inside of the large intestine 28) of the subject 26. Examples of the camera 52 include a CMOS camera. However, this is merely an example, and other types of cameras such as CCD cameras may be used.

The illumination device 54 includes illumination windows 54A and 54B. The illumination windows 54A and 54B are provided on the distal end surface 50A. The illumination device 54 emits the light 30 (see FIG. 1) through the illumination windows 54A and 54B. Examples of the type of the light 30 emitted from the illumination device 54 include light for WLI (for example, white light), light for LCI (for example, light obtained by combining red light, green light, and blue light), light for BLI (for example, blue light), and/or light for NBI (for example, light obtained by combining blue light and green light). The camera 52 images the inside of the large intestine 28 using an optical method in a state in which the inside of the large intestine 28 is irradiated with the light 30 (see FIG. 1) by the illumination device 54.

The treatment tool opening 56 is an opening for allowing a treatment tool 58 to protrude from the distal end portion 50. Moreover, the treatment tool opening 56 is also used as a suction port for suctioning blood, internal contaminants, and the like and a sending-out port for sending out fluid.

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example shown in FIG. 2, an aspect is shown in which a biopsy needle protrudes through the treatment tool opening 56 as the treatment tool 58. Here, the biopsy needle has been described as an example of the treatment tool 58, but this is merely an example, and the treatment tool 58 may be grasping forceps, a papillotomy knife, a snare, a catheter, a guide wire, a cannula, and/or a biopsy needle with a guide sheath.

The endoscope 16 is connected to the control device 20 and the light source device 22 via a universal cord 62. The medical support device 24 and a reception device 64 are connected to the control device 20. Further, the display device 18 is connected to the medical support device 24. Stated another way, the control device 20 is connected to the display device 18 via the medical support device 24.

Here, since the medical support device 24 is used as an example of an external device for expanding the functions of the control device 20, the form example has been described in which the control device 20 and the display device 18 are indirectly connected to each other via the medical support device 24, but this is merely an example. For example, the display device 18 may be directly connected to the control device 20. In such a case, for example, the functions of the medical support device 24 need only be mounted in the control device 20, or the control device 20 need only have a function of directing a server (not shown) to execute the same processing as the processing (for example, the medical support processing which will be described later) executed by the medical support device 24, receiving a processing result obtained by the server, and using the processing result.

The reception device 64 receives an instruction from the doctor 12, and outputs the received instruction as an electric signal to the control device 20. Examples of the reception device 64 include a keyboard, a mouse, a touch panel, a foot switch, a microphone, and/or a remote control device.

The control device 20 controls the light source device 22, transmits and receives various signals to and from the camera 52, or transmits and receives various signals to and from the medical support device 24.

The light source device 22 emits light to supply the light to the illumination device 54 under the control of the control device 20. The illumination device 54 is provided with a built-in light guide, and the light supplied from the light source device 22 is emitted from the illumination windows 54A and 54B via the light guide. The control device 20 causes the camera 52 to perform imaging, acquires the video image 39 (see FIG. 1) from the camera 52, and outputs the video image 39 to a predetermined output destination (for example, the medical support device 24).

The medical support device 24 executes various types of image processing on the video image 39 input from the control device 20 to support the medical treatment (here, for example, endoscopy). The medical support device 24 outputs the video image 39, on which various types of image processing have been performed, to a predetermined output destination (for example, the display device 18).

Here, the form example has been described in which the video image 39 output from the control device 20 is output to the display device 18 via the medical support device 24, but this is merely an example. For example, an aspect may be adopted in which the control device 20 and the display device 18 are connected to each other, and the video image 39 that has been subjected to the image processing by the medical support device 24 is displayed on the display device 18 via the control device 20.

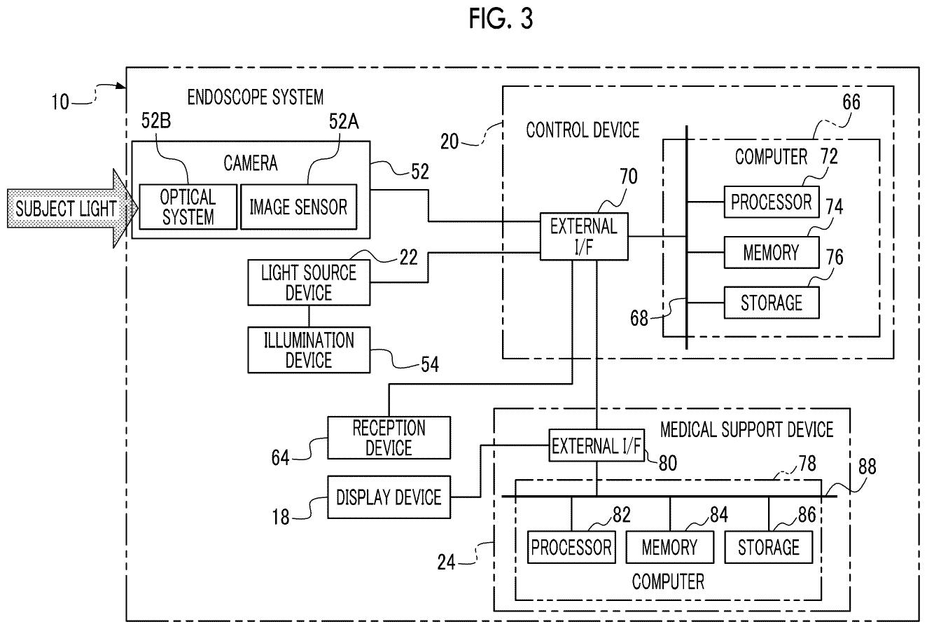

FIG. 3 is a block diagram showing an example of a hardware configuration of an electrical system of the endoscope system 10. As shown in FIG. 3, the control device 20 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 20. The memory 74 and the storage 76 are used by the processor 72.

The external I/F 70 transmits and receives various types of information between one or more devices (hereinafter, also referred to as “first external devices”) existing outside the control device 20 and the processor 72.

The camera 52 is connected to the external I/F 70 as one of the first external devices. The external I/F 70 controls the transmission and the reception of various types of information between the camera 52 and the processor 72. The processor 72 controls the camera 52 through the external I/F 70. In addition, the processor 72 acquires, via the external I/F 70, the video image 39 (see FIG. 1) obtained by imaging the inside of the large intestine 28 (see FIG. 1) via the camera 52.

The camera 52 is provided with an optical system 52B and an image sensor 52A. Examples of the image sensor 52A include a CMOS image sensor and a CCD image sensor. The optical system 52B is an optical system that implements a so-called optical zoom, and operates under the control of the processor 72. The optical system 52B operates to change the magnification of the video image 39 (see FIG. 1). Stated another way, the frame 40 (see FIG. 1) is optically zoomed in or zoomed out.

The image sensor 52A receives the subject light incident on the optical system 52B and photoelectrically converts the received subject light to generate an electric signal in accordance with the subject light. A signal processing circuit (not shown) connected to the image sensor 52A is included in the camera 52. The signal processing circuit acquires the electric signal from the image sensor 52A and executes various types of signal processing including A/D conversion on the acquired electric signal to generate the frame 40 (see FIG. 1) in accordance with a predetermined frame rate (for example, a frame rate determined in advance, such as 15 frames/second, 30 frames/second, or 60 frames/second).

Each time the frame 40 (see FIG. 1) is generated by the camera 52 in accordance with the predetermined frame rate, the generated frame 40 is acquired by the processor 72. Stated another way, the processor 72 acquires the video image 39 (see FIG. 1) including the plurality of frames 40 in time series from the camera 52.

Here, although the form example has been described in which the frame 40 is generated by executing various types of signal processing on the electric signal in accordance with the subject light by the signal processing circuit of the camera 52, this is merely an example. For example, the control device 20 may include a signal processing circuit, and the signal processing circuit of the control device 20 may acquire the electric signal in accordance with the subject light from the image sensor 52A and execute various types of signal processing to generate the frame 40.

The light source device 22 is connected to the external I/F 70 as one of the first external devices, and the external I/F 70 transmits and receives various types of information between the light source device 22 and the processor 72. The light source device 22 supplies the light to the illumination device 54 under the control of the processor 72. The illumination device 54 emits the light supplied from the light source device 22.

The reception device 64 is connected to the external I/F 70 as one of the first external devices, and the processor 72 acquires the instruction received by the reception device 64 via the external I/F 70 and executes the processing corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a “computer” according to the present disclosure, and the processor 82 is an example of a “processor” according to the present disclosure.

In addition, a hardware configuration (that is, the processor 82, the memory 84, and the storage 86) of the computer 78 is essentially the same as the hardware configuration of the computer 66, and thus the description of the hardware configuration of the computer 78 will be omitted here.

The external I/F 80 transmits and receives various types of information between one or more devices (hereinafter, also referred to as “second external devices”) existing outside the medical support device 24 and the processor 82.

The control device 20 is connected to the external I/F 80 as one of the second external devices. In the example shown in FIG. 3, the external I/F 70 of the control device 20 is connected to the external I/F 80. The external I/F 80 transmits and receives various types of information between the processor 82 of the medical support device 24 and the processor 72 of the control device 20. For example, the processor 82 acquires the video image 39 (see FIG. 1) from the processor 72 of the control device 20 via the external I/Fs 70 and 80, and performs various types of image processing on the acquired video image 39.

The display device 18 is connected to the external I/F 80 as one of the second external devices. The processor 82 controls the display device 18 via the external I/F 80 such that various types of information (for example, the video image 39 on which various types of image processing have been performed) are displayed on the display device 18.

In recent years, there has been a development of a technique for recognizing a medical feature of the lesion 42 shown in the frame 40 (that is, the lesion 42 included as an image in the frame 40) using a trained model that has been trained through machine learning for the medical feature of the lesion shown in the image (for example, the endoscopic image). Here, the medical feature refers to a type of the lesion 42 (for example, adenoma, inflammatory polyp, hyperplastic polyp, serrated polyp, lymphoid polyp, adenocarcinoma, mucinous adenocarcinoma, sclerosing adenocarcinoma, squamous cell carcinoma, and malignant lymphoma), morphological types of lesion 42 (for example, pedunculated, semi-pedunculated, sessile, surface-elevated type, surface-flat type, and surface-depressed type), and/or the malignancy degree of the lesion 42.

For example, the recognition result obtained by the trained model, that is, the medical feature is displayed on the screen 35 or the like. The doctor 12 can perform an appropriate treatment on the subject 26 by referring to the medical feature displayed on the screen 35 or the like.

However, in a case in which the image input to the trained model is a non-sharp image due to blurriness or the like, it is difficult to obtain a highly reliable recognition result from the trained model.

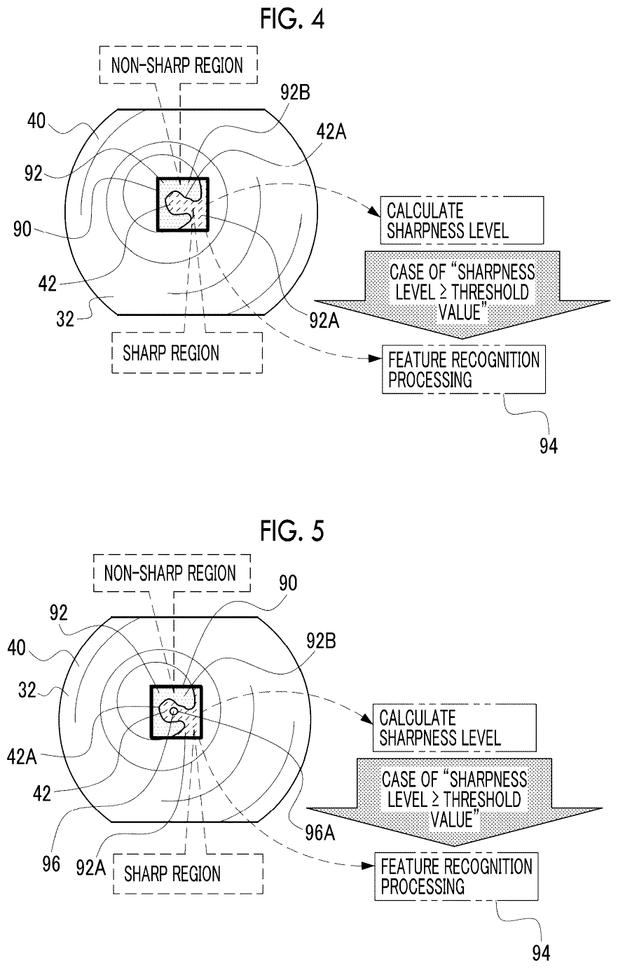

Therefore, in the related art, the AI-based recognition processing is performed on the image of interest only in a case in which a degree of blurriness of the image of interest in which the lesion 42 is shown in the frame 40 is less than a reference level. Hereinafter, an outline of a known technique in the related art will be described with reference to FIGS. 4 to 6.

In the examples shown in FIGS. 4 to 6, the lesion 42 shown in the frame 40 is recognized by a bounding box method by executing AI-based object recognition processing. Stated another way, by executing the object recognition processing on the frame 40, an image region in which the lesion 42 in the frame 40 is shown is surrounded by a bounding box 90 (in the examples shown in FIGS. 4 to 6, a rectangular frame) as an image of interest 92.

In the example shown in FIG. 4, the image of interest 92 includes a sharp region 92A which is a sharp image region, and a non-sharp region 92B which is a non-sharp image region. Examples of the sharp region 92A include an image region in focus. Examples of the non-sharp region 92B include an image region in which the focus is not aligned (for example, an image region showing an aspect on the front side with respect to the sharp region 92A and/or an image region showing an aspect on the background side). Here, the image region in focus has been described as an example of the sharp region 92A, and the image region out of focus has been described as an example of the non-sharp region 92B, but this is merely an example, and the sharp region 92A may be an image region within a depth of field, and the non-sharp region 92B may be an image region out of the depth of field.

In the example shown in FIG. 4, in the image of interest 92, the lesion 42 shown in the frame 40 is in focus, while the image region other than the lesion 42 is not in focus. Stated another way, in the image of interest 92, a high-frequency component of the sharp region 92A is larger than a high-frequency component of the non-sharp region 92B. In the example shown in FIG. 4, an outer edge 42A of the lesion 42 (that is, an edge of the lesion 42) in the image of interest 92 is a boundary between the sharp region 92A and the non-sharp region 92B, and has the largest high-frequency component in the image of interest 92.

In the example shown in FIG. 4, a sharpness level of the image of interest 92 is calculated. Examples of the method of calculating the sharpness level of the image of interest 92 include a calculation method in accordance with the following steps (1) to (3). First, in step (1), the edge such as the outer edge 42A is enhanced by applying a Laplacian filter to the image of interest 92. Next, in step (2), a total value (hereinafter, simply referred to as a “total value”) of absolute values of pixel values of pixels of the image of interest 92 to which the Laplacian filter is applied is calculated. In step (3), the total value is divided by the number of pixels of the image of interest 92. The division result obtained in step (3) means an average value of the intensities of the edges of the image of interest 92.

The division result obtained in step (3) (that is, the average value) is the sharpness level of the image of interest 92. A higher division result obtained in step (3) means that the image of interest 92 is sharper, and a lower division result obtained in step (3) means that the image of interest 92 is less sharp.

As shown in FIG. 4, in a case in which the sharpness level of the image of interest 92 is equal to or greater than a threshold value, feature recognition processing 94 is executed on the image of interest 92. The feature recognition processing 94 is processing of recognizing the medical feature by using an AI-based method (that is, recognition processing using a trained model that has been trained through machine learning for the medical feature of the lesion shown in the image). The processing result obtained by executing the feature recognition processing 94 on the image of interest 92 or information based on the processing result is provided to the doctor 12 via the screen 35 or the like.

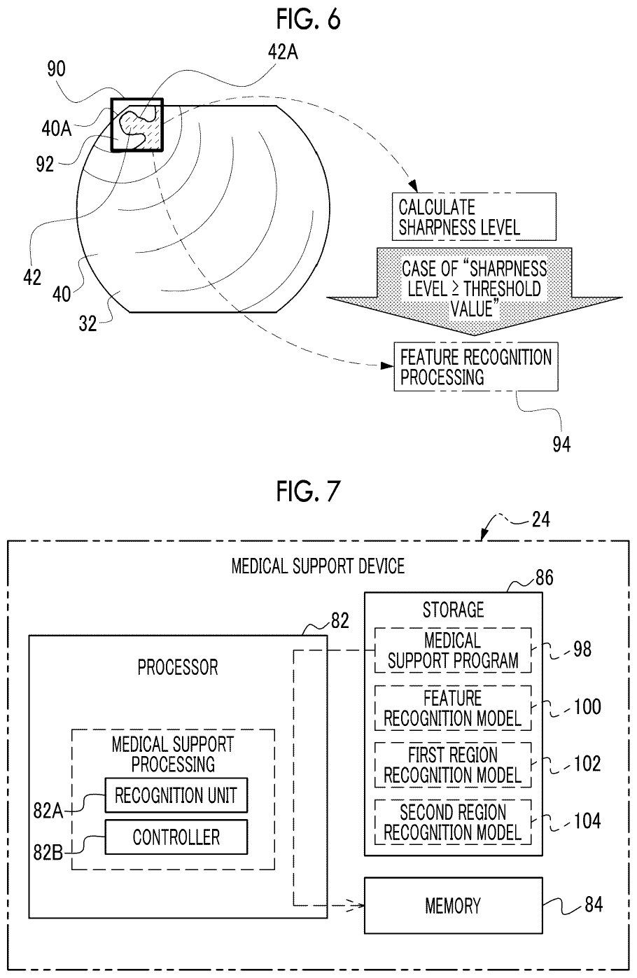

However, in a case in which the outer edge 42A (that is, the outer edge 42A included as an image in the image of interest 92) included in the image of interest 92 is enhanced by the Laplacian filter, a condition of “sharpness level of image of interest 92≥threshold value” may be satisfied even though most of the image of interest 92 is the non-sharp region 92B. In addition, as shown in FIG. 5 as an example, an edge 96A of a halation portion 96 (in other words, a light reflection portion or a gloss portion) generated by irradiating the lesion 42 (that is, the lesion 42 included as an image in the image of interest 92) shown in the image of interest 92 with the light 30 is also enhanced by the Laplacian filter. In addition, as shown in FIG. 6 as an example, in a case in which an edge 40A (that is, the outer edge) of the frame 40 is shown in the image of interest 92, the edge 40A is enhanced by the Laplacian filter. The edges 40A and 96A work as factors satisfying the condition of “sharpness level of the image of interest 92≥threshold value” even though the edges 40A and 96A are unnecessary information for recognizing the medical feature of the lesion 42 by the feature recognition processing 94.

Then, since the feature recognition processing 94 is executed on the image of interest 92 even though the image of interest 92 is an image having the sharpness level in which the medical feature is erroneously recognized, there is a concern that the processing result having low reliability by the feature recognition processing 94 or the information based on the processing result having low reliability by the feature recognition processing 94 may be provided to the doctor 12 or the like.

Therefore, in view of such circumstances, in the present embodiment, for example, as shown in FIG. 7, the processor 82 of the medical support device 24 executes the medical support processing.

A medical support program 98 is stored in the storage 86. The medical support program 98 is an example of a “program” according to the present disclosure. The processor 82 reads out the medical support program 98 from the storage 86, and executes the readout medical support program 98 on the memory 84 to perform the medical support processing. The medical support processing is implemented by the processor 82 operating as a recognition unit 82A and a controller 82B in accordance with the medical support program 98 executed on the memory 84.

The storage 86 stores a feature recognition model 100, a first region recognition model 102, and a second region recognition model 104. Although details will be described later, the feature recognition model 100, the first region recognition model 102, and the second region recognition model 104 are used by the recognition unit 82A.

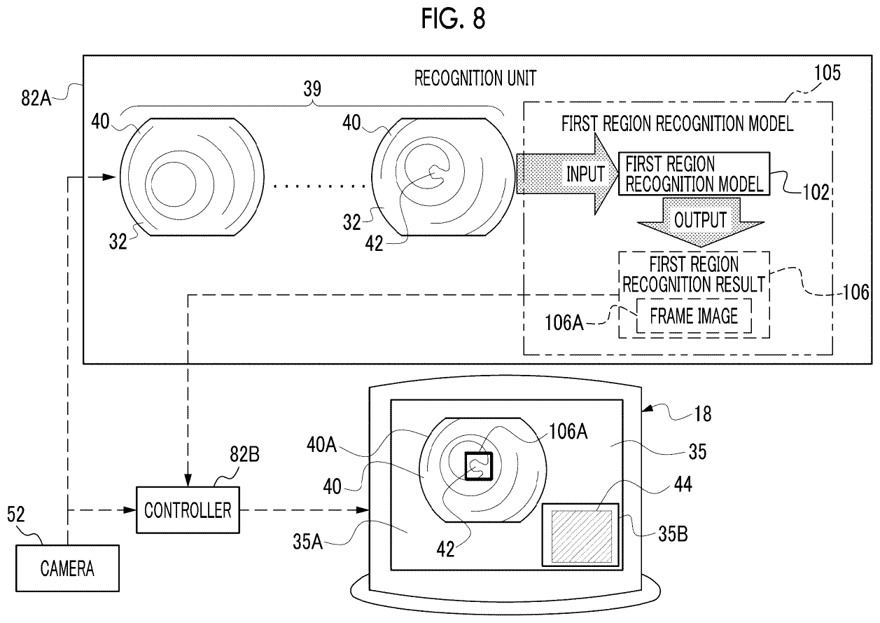

For example, as shown in FIG. 8, the recognition unit 82A and the controller 82B acquire each of the plurality of frames 40, which are arranged in time series in the video image 39 generated by being captured by the camera 52 in accordance with an imaging frame rate (for example, several tens of frames/second), from the camera 52 frame by frame in time series. Timings at which the recognition unit 82A and the controller 82B acquire the frame 40 are synchronized with each other.

The controller 82B outputs the video image 39 to the display device 18. For example, the controller 82B displays the video image 39 in the first display region 35A as a live view image. Stated another way, the controller 82B displays the acquired frame 40 in the first display region 35A in order in accordance with the predetermined frame rate each time the frame 40 is acquired from the camera 52. Furthermore, the controller 82B displays the auxiliary information 44 in the second display region 35B. Furthermore, the controller 82B updates the display content (for example, the auxiliary information 44) of the second display region 35B according to the display content of the first display region 35A.

The recognition unit 82A recognizes the lesion 42 in the video image 39 based on the video image 39 acquired from the camera 52. Stated another way, the recognition unit 82A sequentially performs first region recognition processing 105 on each of the plurality of frames 40 arranged in time series in the video image 39 acquired from the camera 52, to recognize the lesion 42 shown in the frame 40.

The first region recognition processing 105 is performed on the acquired frame 40 each time the recognition unit 82A acquires the frame 40. The first region recognition processing 105 is processing of recognizing the lesion 42 by using an AI-based method. Here, the processing using the first region recognition model 102 is performed as the first region recognition processing 105.

The first region recognition model 102 is a trained model for object recognition in a bounding box method using AI. In addition, the first region recognition model 102 is a trained model obtained by optimizing a neural network by training the neural network through machine learning using first training data. The first training data is a dataset including a plurality of data (that is, data for a plurality of frames) in which first example data is associated with first ground truth data.

The first example data is an image that assumes the frame 40. A first example of the image that assumes the frame 40 is an image obtained by actually imaging the inside of the large intestine with the camera. A second example of the image that assumes the frame 40 is an image virtually created (for example, an image generated by generative AI). The first ground truth data is ground truth data (that is, an annotation) for the first example data. Here, examples of the first ground truth data include position information (for example, coordinates) indicating a position of a rectangular region surrounding an image region including the lesion shown in the image used as the first example data.

The recognition unit 82A acquires the frame 40 from the camera 52, and inputs the acquired frame 40 to the first region recognition model 102. As a result, the first region recognition model 102 recognizes the image region including the lesion 42 shown in the input frame 40 each time the frame 40 is input, generates a first region recognition result 106 that is the recognition result, and outputs the first region recognition result 106. The first region recognition result 106 includes a frame image 106A indicating an outer edge of the rectangular region specified from the position information, in addition to the position information indicating the position of the rectangular region surrounding the image region including the lesion 42. The frame image 106A corresponds to the bounding box. Stated another way, the geometrical characteristics (for example, the size, the position, and the shape) of the frame image 106A match the geometrical characteristics of the bounding box.

The controller 82B displays the frame image 106A in a superimposed manner on the frame 40 displayed in the first display region 35A based on the first region recognition result 106 input from the recognition unit 82A. As a result, the lesion 42 shown in the frame 40 displayed in the first display region 35A is surrounded by the frame image 106A.

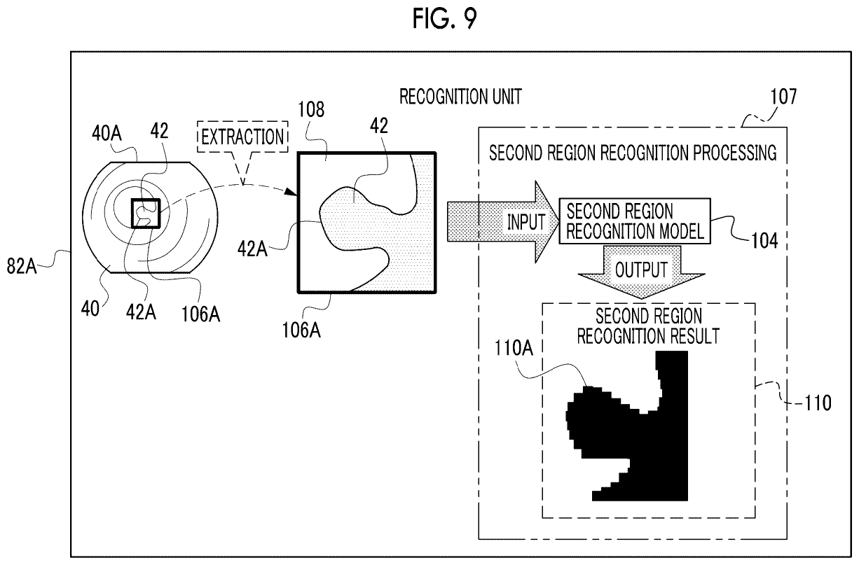

As shown in FIG. 9 as an example, the recognition unit 82A extracts a processing target image 108 that is a portion surrounded by the frame image 106A, from the same frame 40 as the frame 40 input to the first region recognition model 102 in order to obtain the first region recognition result 106 including the frame image 106A. The processing target image 108 shows the lesion 42 and the background side (in the example shown in FIG. 9, the lumen) of the lesion 42. Since the lesion 42 is shown in the processing target image 108, the processing target image 108 can also be referred to as an image of interest that is an image of interest of the doctor 12.

The recognition unit 82A recognizes the lesion 42 shown in the processing target image 108 (that is, the lesion 42 included as an image in the processing target image 108) by performing second region recognition processing 107 on the processing target image 108. The second region recognition processing 107 in the present embodiment is an example of “region recognition processing” according to the present disclosure.

The second region recognition processing 107 is performed on each processing target image 108 extracted from the same frame 40 as the frame 40 on which the first region recognition processing 105 is performed. The second region recognition processing 107 is processing of recognizing the lesion 42 by using an AI-based method. As the second region recognition processing 107, the processing using the second region recognition model 104 is performed.

The second region recognition model 104 is a trained model for object recognition by an AI-based segmentation method. The second region recognition model 104 is a trained model obtained by optimizing a neural network by training the neural network through machine learning using second training data. The second training data is a dataset including a plurality of data (that is, data for a plurality of frames) in which the second example data is associated with the second ground truth data.

The second example data is an image that assumes the processing target image 108. A first example of the image that assumes the processing target image 108 is an image in which an image region including a lesion shown in an image obtained by actually imaging the inside of the large intestine with a camera is rectangularly cut out. A second example of the image that assumes the processing target image 108 is a virtually generated image (for example, an image generated by generative AI). The second ground truth data is ground truth data (that is, an annotation) for the second example data. Here, examples of the second ground truth data include a lesion label added to each pixel of the image region indicating the lesion shown in the image used as the second example data. The lesion label refers to a label indicating the lesion.

The recognition unit 82A inputs the processing target image 108 to the second region recognition model 104. As a result, the second region recognition model 104 recognizes the image region showing the lesion 42 shown in the input processing target image 108 in units of pixels each time the processing target image 108 is input, generates a second region recognition result 110 that is the recognition result, and outputs the second region recognition result 110. The second region recognition result 110 includes a segmentation mask 110A capable of specifying the position of the image region indicating the lesion 42 in the processing target image 108, in addition to the position information (for example, coordinates) indicating the position of each pixel of the image region indicating the lesion 42 in the processing target image 108. The segmentation mask 110A is a mask generated by a segmentation algorithm (for example, a U-net or a Mask R-CNN).

A resolution of the segmentation mask 110A is lower than a resolution of the frame 40. Each pixel of the segmentation mask 110A is associated with the position information (for example, coordinates) indicating the corresponding position in the processing target image 108 and a confidence level indicating that the lesion 42 is present. The segmentation mask 110A in the present embodiment is an example of a “segmentation mask” according to the present disclosure.

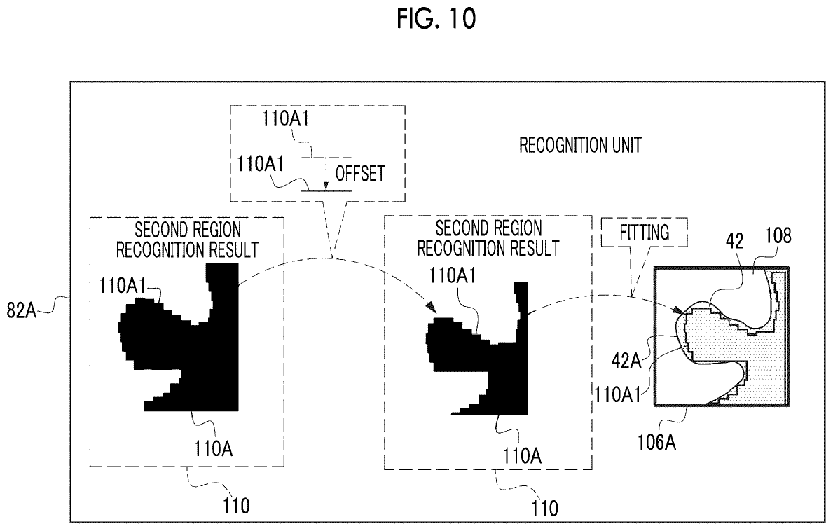

As shown in FIG. 10 as an example, the recognition unit 82A reduces an outer shape of the segmentation mask 110A by offsetting an outer edge 110A1 of the segmentation mask 110A to the inside of the segmentation mask 110A. In the segmentation mask 110A, the confidence level (in other words, the probability indicating that the lesion 42 is present or an indicator indicating a likelihood indicating that the lesion 42 is present) on the outer edge 110A1 after being offset to the inside of the segmentation mask 110A is higher than the confidence level on the outer edge 110A1 before being offset to the inside of the segmentation mask 110A. This is because the outer edge 110A1 after being offset to the inside of the segmentation mask 110A is farther from the region outside the lesion 42 and is closer to the region having a high degree of the likelihood of the lesion 42 (that is, a high confidence level) than the outer edge 110A1 before being offset to the inside of the segmentation mask 110A.

Examples of a direction for offsetting the outer edge 110A1 include a direction toward the center of a circumscribed rectangular region of the segmentation mask 110A or a centroid of the segmentation mask 110A. In addition, examples of an offset amount for offsetting the outer edge 110A1 include a predetermined amount (for example, the number of pixels) as the offset amount for positioning the outer edge 110A1 inside the outer edge 42A of the lesion 42 shown in the processing target image 108. Here, although the form example has been described in which the outer edge 110A1 is offset, this is merely an example, and the outer shape of the segmentation mask 110A may be reduced by performing contraction processing (for example, contraction processing of morphology conversion) on the segmentation mask 110A.

The recognition unit 82A fits the outer edge 110A1 of the segmentation mask 110A having the reduced outer shape to the processing target image 108. Stated another way, the recognition unit 82A maps the pixel of the outer edge 110A1 of the segmentation mask 110A to the corresponding position in the processing target image 108. Here, the corresponding position in the processing target image 108 means a position indicated by the position information associated with each pixel of the outer edge 110A1 of the segmentation mask 110A. As described above, in a case in which the outer edge 110A1 of the segmentation mask 110A having the reduced outer shape is fitted to the processing target image 108, the outer edge 110A1 of the segmentation mask 110A is located inside the outer edge 42A of the lesion 42.

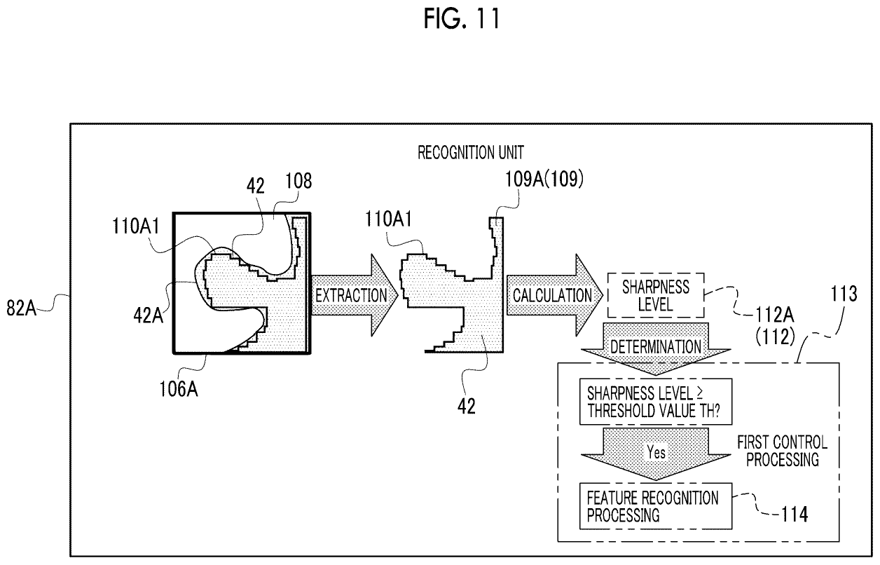

As shown in FIG. 11 as an example, the recognition unit 82A extracts a region based on the segmentation mask 110A as an inner region 109A from the processing target image 108. The inner region 109A is an image region inside the outer edge 42A of the lesion 42, which is shown in the processing target image 108, in the processing target image 108. The image region inside the outer edge 42A of the lesion 42 shown in the processing target image 108 in the processing target image 108 means an image region inside the outer edge 110A1 of the segmentation mask 110A having the reduced outer shape among all image regions of the processing target image 108. In the example shown in FIG. 11, the recognition unit 82A extracts an image of a portion surrounded by the outer edge 110A1 in the processing target image 108 as the inner region 109A from the processing target image 108. The outer edge 42A of the lesion 42 is not included in the inner region 109A. This means that the high-frequency component at the same level as the outer edge 42A is not included in the inner region 109A. The inner region 109A in the present embodiment is an example of an “inner region” according to the present disclosure.

The recognition unit 82A calculates a sharpness level 112A of the inner region 109A. The method of calculating the sharpness level 112A of the inner region 109A is the same as the method of calculating the sharpness level of the image of interest 92 in the example shown in FIGS. 4 to 6. The sharpness level 112A can be said to be a sharpness level having higher reliability than the sharpness level 112A of the processing target image 108. This is because the processing target image 108 includes an image region corresponding to the non-sharp region 92B shown in FIG. 4, and thus, in a case in which the sharpness level of the processing target image 108 is calculated, an influence of an edge component (that is, the high-frequency component) of the outer edge 42A or the like of the lesion 42 is reflected in the calculation result, whereas the sharpness level 112A of the inner region 109A is hardly affected by the influence of the outer edge 42A (that is, the high-frequency component) of the lesion 42.

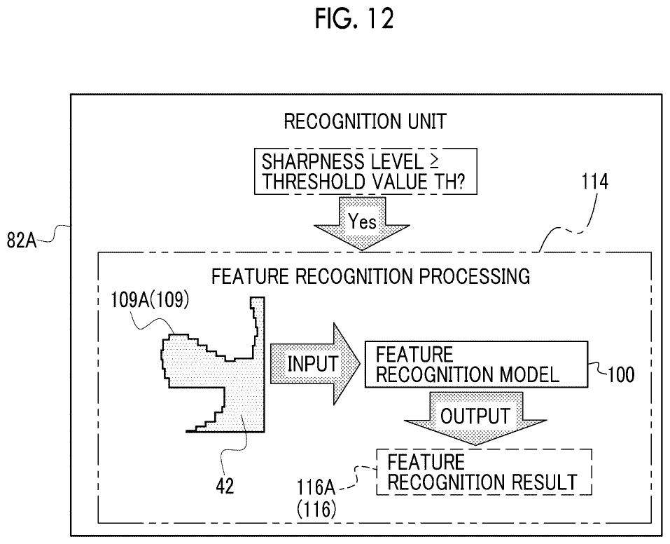

The recognition unit 82A performs first control processing 113 in accordance with the sharpness level 112A. The first control processing 113 is processing of controlling feature recognition processing 114. In the first control processing 113, it is determined whether or not the sharpness level 112A is equal to or greater than a threshold value TH. The threshold value TH is a value derived in advance by a test with an actual machine and/or a computer simulation as a lower limit value of the sharpness level 112A at which the medical feature of the lesion 42 (that is, the lesion 42 shown in the inner region 109A as an image) shown in the inner region 109A is not erroneously recognized by the feature recognition model 100 by inputting the inner region 109A to the feature recognition model 100 (see FIGS. 7 and 12).

The first control processing 113 includes processing of performing the feature recognition processing 114 by the recognition unit 82A in a case in which the sharpness level 112A is equal to or greater than the threshold value TH and not performing the feature recognition processing 114 by the recognition unit 82A in a case in which the sharpness level 112A is less than the threshold value TH.

The first control processing 113 in the present embodiment is an example of “first processing” according to the present disclosure. In addition, the feature recognition processing 114 in the present embodiment is an example of “image recognition processing” according to the present disclosure. In addition, the threshold value TH in the present embodiment is an example of a “first threshold value” and a “second threshold value” according to the present disclosure.

As shown in FIG. 12 as an example, the feature recognition processing 114 is performed on the frame 40 by the recognition unit 82A. In the example shown in FIG. 12, an aspect is shown in which the recognition unit 82A performs processing using the feature recognition model 100 as the feature recognition processing 114 on the inner region 109A. The feature recognition processing 114 is processing of recognizing the feature of the lesion 42 by using an AI-based method. Here, the feature of the lesion 42 means a medical feature of the lesion 42.

The feature recognition model 100 is a trained model for object recognition. In addition, the feature recognition model 100 is a trained model obtained by optimizing a neural network by training the neural network through machine learning using third training data. The third training data is a dataset including a plurality of data (that is, data for a plurality of frames) in which third example data is associated with third ground truth data.

The third example data is an image that assumes the image (for example, the inner region 109A) showing the lesion 42. A first example of the image that assumes the lesion 42 is an image obtained by actually imaging the lesion in the large intestine with the camera. A second example of the image that assumes the image showing the lesion 42 is a virtually generated image (for example, an image generated by generative AI). The third ground truth data is ground truth data (that is, an annotation) for the third example data. Here, examples of the third ground truth data include information indicating the medical feature of the lesion indicated by the image used as the third example data. Examples of the medical feature of the lesion indicated by the image used as the third example data include the type of the lesion indicated by the image used as the third example data, the morphological type of the lesion indicated by the image used as the third example data, and/or the malignancy degree of the lesion indicated by the image used as the third example data.

The recognition unit 82A inputs the inner region 109A to the feature recognition model 100. As a result, the feature recognition model 100 recognizes the medical feature of the lesion 42 indicated by the input inner region 109A each time the inner region 109A is input, generates a feature recognition result 116A that is the recognition result, and outputs the feature recognition result 116A.

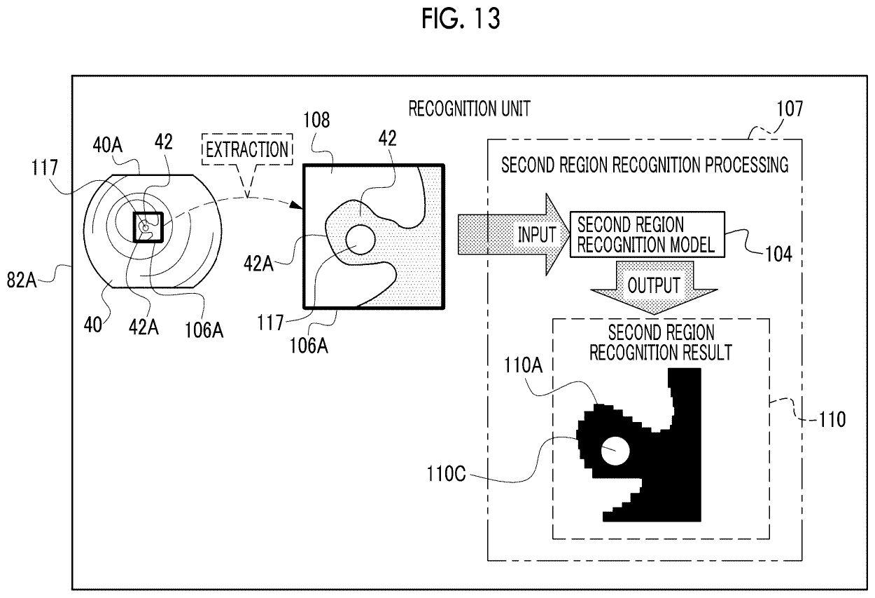

As shown in FIG. 13 as an example, the processing target image 108 may include a halation portion 117 together with the lesion 42. The halation portion 117 means a portion (in other words, a light reflection portion or a gloss portion) in which halation occurs due to the reflection of the light 30 from air bubbles or the like attached to the lesion 42. In a case in which the halation portion 117 is present in the lesion 42, the segmentation mask 110A included in the second region recognition result 110 has a blank region 110C corresponding to the halation portion 117. A position of the blank region 110C in the segmentation mask 110A corresponds to the position of the halation portion 117 in the processing target image 108.

Here, in a case in which the sharpness level of the processing target image 108 in which the halation portion 117 is shown is calculated by the above-described calculation method, the influence of the edge component (that is, the high-frequency component) of the halation portion 117 is reflected in the calculation result, and the reliability of the sharpness level of the processing target image 108 is decreased.

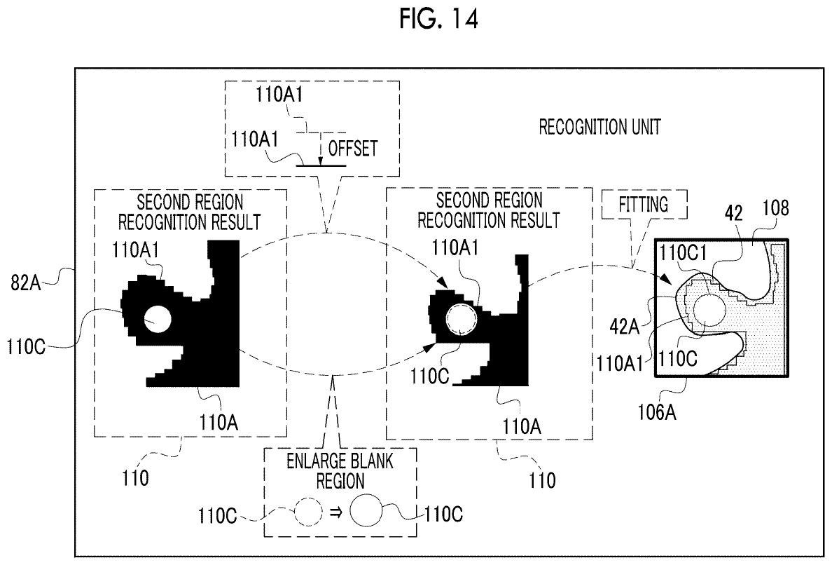

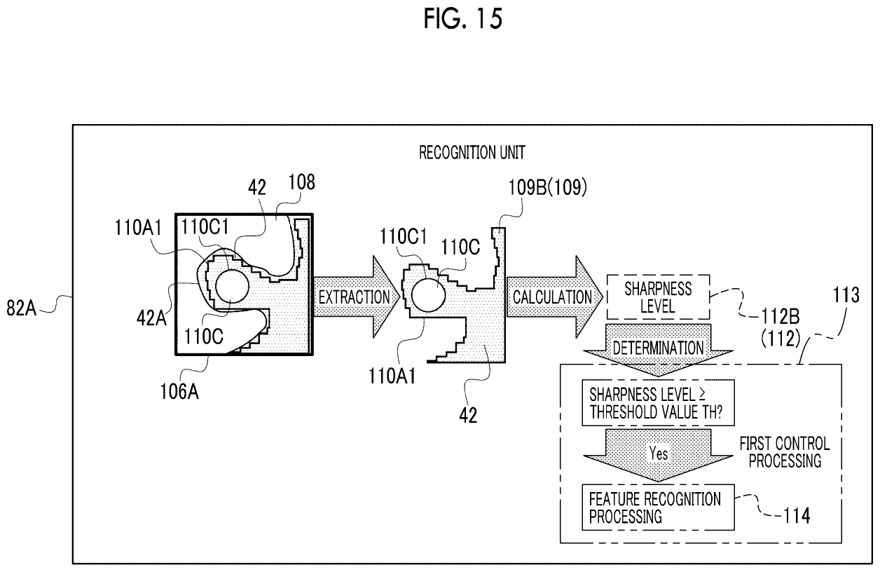

Therefore, in the present embodiment, as an example, as shown in FIGS. 14 and 15, the recognition unit 82A extracts an inner region 109B in which the halation portion 117 is considered, from the processing target image 108.

In such a case, first, as shown in FIG. 14 as an example, the recognition unit 82A offsets the outer edge 110A1 of the segmentation mask 110A and enlarges the blank region 110C of the segmentation mask 110A in the same method as in the example shown in FIG. 10. A enlargement ratio used for enlarging the blank region 110C is determined in accordance with the geometrical characteristics (for example, the shape, the size, and the like) of the segmentation mask 110A and the geometrical characteristics (for example, the shape, the position, the size, and the like) of the blank region 110C in the segmentation mask 110A. The blank region 110C is a region outside the edge of the blank region 110C before the enlargement and is enlarged to a region inside the outer edge 110A1 of the segmentation mask 110A.

In the segmentation mask 110A, the confidence level of the region outside the edge of the blank region 110C before the enlargement and inside the outer edge 110A1 of the segmentation mask 110A is higher than the confidence level on the edge of the blank region 110C before the enlargement in the segmentation mask 110A. This is because, in the segmentation mask 110A, the region outside the edge of the blank region 110C before the enlargement and inside the outer edge 110A1 of the segmentation mask 110A is farther away from the halation portion 117, which is is a region different from the lesion 42, and is closer to a region having a high degree of the likelihood of the lesion 42 (that is, a high confidence level) than the edge of the blank region 110C before the enlargement in the segmentation mask 110A.

The recognition unit 82A fits the outer edge 110A1 of the segmentation mask 110A having the reduced outer shape and an edge 110C1 of the enlarged blank region 110C to the processing target image 108. Stated another way, the recognition unit 82A maps the pixel of the outer edge 110A1 to the corresponding position in the processing target image 108 and maps the pixel of the edge 110C1 to the corresponding position in the processing target image 108. Here, the position to which the pixel of the outer edge 110A1 is mapped is the position indicated by the position information associated with each pixel of the outer edge 110A1 of the segmentation mask 110A. In addition, the position to which the pixel of the edge 110C1 is mapped is the position indicated by the position information associated with each pixel of the edge 110C1 of the segmentation mask 110A.

As shown in FIG. 15 as an example, the recognition unit 82A extracts the inner region 109B from the processing target image 108. The inner region 109B is a region inside the outer edge 42A of the lesion 42 shown in the processing target image 108 in the processing target image 108 and is a region outside the edge of the halation portion 117. In the example shown in FIG. 15, an image of a portion surrounded by the outer edge 110A1 and the edge 110C1 in the processing target image 108 is extracted from the processing target image 108, as the inner region 109B.

The inner region 109B in the present embodiment is an example of an “inner region” according to the present disclosure. In addition, the halation portion 117 in the present embodiment is an example of a “halation portion” according to the present disclosure. In addition, the edge of the halation portion 117 in the present embodiment is an example of an “edge of the halation portion” according to the present disclosure.

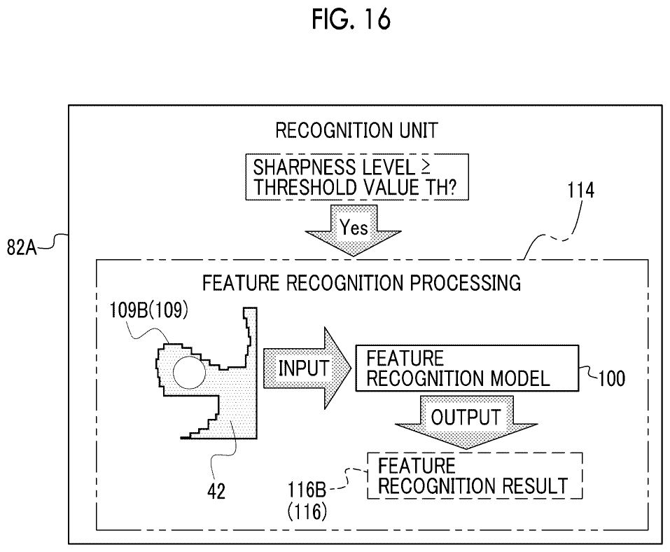

The recognition unit 82A calculates a sharpness level 112B of the inner region 109B in the same method as in the calculation of the sharpness level 112A of the inner region 109A. The recognition unit 82A performs the first control processing 113 using the sharpness level 112B in the same method as the first control processing 113 is performed using the calculated sharpness level 112A. Then, in a case in which the sharpness level 112B is equal to or greater than the threshold value TH, as shown in FIG. 16 as an example, the recognition unit 82A inputs the inner region 109B to the feature recognition model 100. As a result, the feature recognition model 100 recognizes the medical feature of the lesion 42 indicated by the input inner region 109B each time the inner region 109B is input, generates a feature recognition result 116B that is the recognition result, and outputs the feature recognition result 116B.

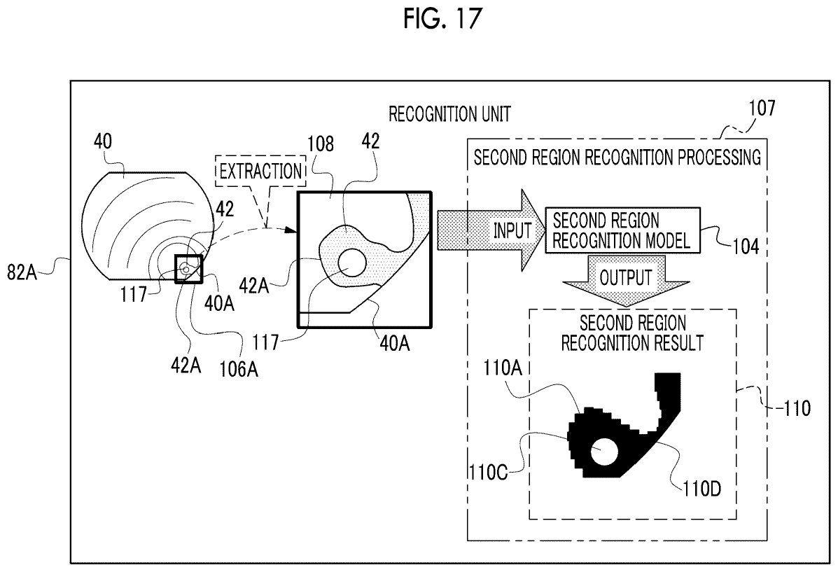

As shown in FIG. 17 as an example, in a case in which a part of the lesion 42 shown in the frame 40 deviates from the frame 40 (in other words, in a case in which a part of the lesion 42 deviates from the frame), the edge 40A of the frame 40 is shown in the processing target image 108 together with the lesion 42. In a case in which the edge 40A of the frame 40 is shown together with the lesion 42 in the processing target image 108, the segmentation mask 110A has an edge 110D. A position of the edge 110D corresponds to the position of the edge 40A in the processing target image 108.

Here, in a case in which the sharpness level of the processing target image 108 in which the edge 40A is shown is calculated by the above-described calculation method, the influence of the edge 40A (that is, the high-frequency component) is reflected in the calculation result, and the reliability of the sharpness level of the processing target image 108 is decreased.

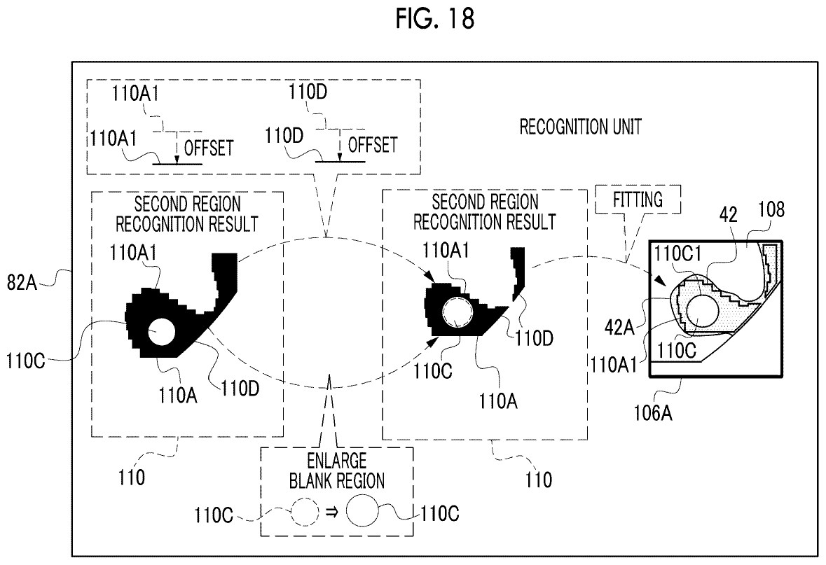

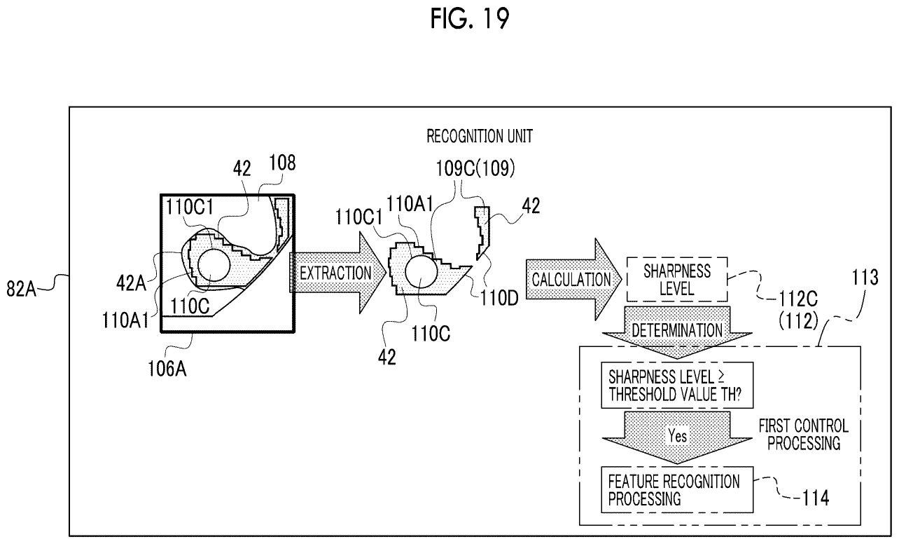

In the present embodiment, as an example, as shown in FIGS. 18 and 19, the recognition unit 82A offsets the outer edge 110A1 of the segmentation mask 110A to the inside of the segmentation mask 110A in the same method as in the example shown in FIG. 10, enlarges the blank region 110C of the segmentation mask 110A in the same method as in the example shown in FIG. 14, and offsets the edge 110D of the segmentation mask 110A to the inside of the segmentation mask 110A. As a result, the outer shape of the segmentation mask 110A is reduced, and an occupancy of the blank region 110C in the segmentation mask 110A is increased.

In the segmentation mask 110A, the confidence level on the edge 110D after being offset to the inside of the segmentation mask 110A is higher than the confidence level on the edge 110D before being offset to the inside of the segmentation mask 110A. This is because the edge 110D after being offset to the inside of the segmentation mask 110A is farther away from a region (for example, a region outside the frame 40) different from the lesion 42 and is closer to a region having a high degree of likelihood of the lesion 42 (that is, a high confidence level) than the edge 110D before being offset to the inside of the segmentation mask 110A.

Examples of a direction for offsetting the edge 110D include a direction toward the center of the circumscribed rectangular region of the segmentation mask 110A or the centroid of the segmentation mask 110A. In addition, examples of the offset amount for offsetting the edge 110D include an amount determined in advance as the offset amount for positioning the edge 110D inside the edge 40A included in the processing target image 108 (that is, the edge 40A included as an image in the processing target image 108) and inside the outer edge 42A of the lesion 42.

Here, although the form example has been described in which the edge 110D is offset, this is merely an example, and the outer shape of the segmentation mask 110A including the edge 110D may be reduced by performing contraction processing (for example, contraction processing of morphology conversion) on the segmentation mask 110A.

The recognition unit 82A fits the outer edge 110A1 of the segmentation mask 110A having the reduced outer shape, the edge 110D of the segmentation mask 110A having the reduced outer shape, and the edge 110C1 of the blank region 110C to the processing target image 108. Stated another way, the recognition unit 82A maps the pixel of the outer edge 110A1 to the corresponding position in the processing target image 108, maps the pixel of the edge 110C1 to the corresponding position in the processing target image 108, and maps the pixel of the edge 110D to the corresponding position in the processing target image 108.

The position to which the pixel of the outer edge 110A1 is mapped is the position indicated by the position information associated with each pixel of the outer edge 110A1 of the segmentation mask 110A. In addition, the position to which the pixel of the edge 110C1 is mapped is the position indicated by the position information associated with each pixel of the edge 110C1 of the segmentation mask 110A. In addition, the position to which the pixel of the edge 110D is mapped is the position indicated by the position information associated with each pixel of the edge 110D of the segmentation mask 110A.

As described above, in a case in which the outer edges 110A1 and 110D of the segmentation mask 110A having the reduced outer shape are fitted to the processing target image 108, the outer edges 110A1 and 110D of the segmentation mask 110A are located inside the lesion 42 with respect to the outer edge 42A and the edge 40A of the lesion 42.

As shown in FIG. 19 as an example, the recognition unit 82A extracts an inner region 109C from the processing target image 108. The inner region 109C is a region (in other words, a region in the processing target image 108 excluding the outer edge 42A and the edge 40A of the lesion 42 shown in the processing target image 108) inside the outer edge 42A and the edge 40A of the lesion 42, which is shown in the processing target image 108, in the processing target image 108, and is a region outside the edge 110C1 of the blank region 110C in the processing target image 108. In the example shown in FIG. 19, an image of a portion surrounded by the outer edge 110A1, the edge 110D, and the edge 110C1 of the blank region 110C in the processing target image 108 is extracted from the processing target image 108, as the inner region 109C. The inner region 109C in the present embodiment is an example of an “inner region” according to the present disclosure.

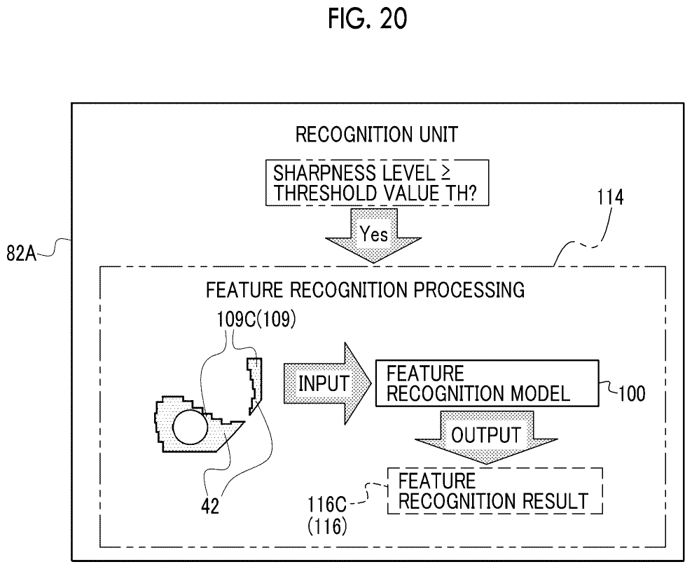

The recognition unit 82A calculates a sharpness level 112C of the inner region 109C in the same method as in the calculation of the sharpness level 112A of the inner region 109A. The recognition unit 82A performs the first control processing 113 using the sharpness level 112C in the same method as the first control processing 113 is performed using the calculated sharpness level 112A. Then, in a case in which the sharpness level 112C is equal to or greater than the threshold value TH, as shown in FIG. 20 as an example, the recognition unit 82A inputs the inner region 109C to the feature recognition model 100. As a result, the feature recognition model 100 recognizes the medical feature of the lesion 42 indicated by the input inner region 109C each time the inner region 109C is input, generates a feature recognition result 116C that is the recognition result, and outputs the feature recognition result 116C.

Hereinafter, for convenience of description, in a case in which it is not necessary to distinguish between the inner regions 109A, 109B, and 109C, the inner regions 109A, 109B, and 109C will be referred to as an “inner region 109”. In addition, in the following description, for convenience of description, in a case in which it is not necessary to distinguish the sharpness levels 112A, 112B, and 112C, the sharpness levels 112A, 112B, and 112C will be collectively referred to as a “sharpness level 112”. In addition, in the following description, for convenience of description, in a case in which it is not necessary to distinguish between the feature recognition results 116A, 116B, and 116C, the feature recognition results 116A, 116B, and 116C will be collectively referred to as a “feature recognition result 116”. In addition, in the following description, for convenience of description, in a case in which it is not necessary to distinguish between the processing of offsetting the outer edge 110A1 of the segmentation mask 110A to the inside of the segmentation mask 110A, the processing of enlarging the blank region 110C in the segmentation mask 110A, and the processing of offsetting the edge 110D of the segmentation mask 110A to the inside of the segmentation mask 110A, the processing is referred to as “processing of fitting the contour of the segmentation mask 110A to the lesion 42 on the processing target image 108”.

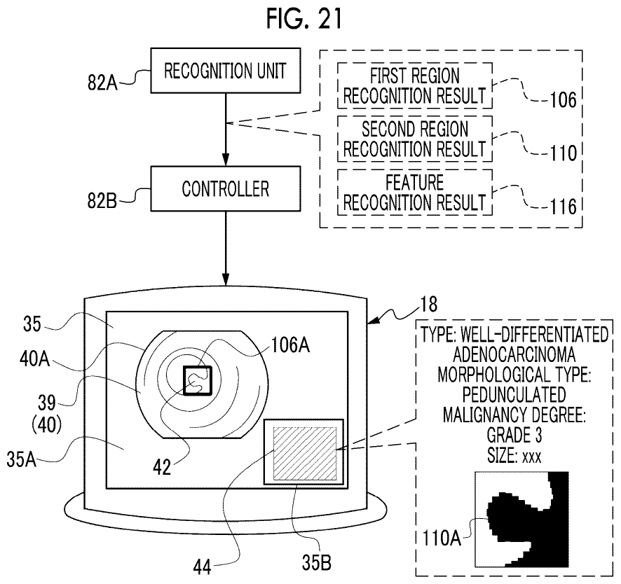

As shown in FIG. 21 as an example, the controller 82B acquires the first region recognition result 106, the second region recognition result 110, and the feature recognition result 116, from the recognition unit 82A. Then, the controller 82B outputs information based on the first region recognition result 106, information based on the second region recognition result 110, and information based on the feature recognition result 116. Examples of the output destination of the information based on the first region recognition result 106, the information based on the second region recognition result 110, and the information based on the feature recognition result 116 include the screen 35. Other examples of the output destination include the storage 76, the storage 86, a server, a personal computer, and/or a tablet terminal.

The output timings of the information based on the first region recognition result 106, the information based on the second region recognition result 110, and the information based on the feature recognition result 116 are timings in accordance with the display timing of the frame 40 in the first display region 35A. For example, the information based on the first region recognition result 106, the information based on the second region recognition result 110, and the information based on the feature recognition result 116 are output in synchronization with the frame 40 displayed in the first display region 35A.

The feature recognition result 116 in the present embodiment is an example of a “processing result of the image recognition processing” according to the present disclosure. In addition, the information based on the feature recognition result 116 in the present embodiment is an example of “information based on a processing result of the image recognition processing” according to the present disclosure. In addition, the screen 35 in the present embodiment is an example of a “screen” according to the present disclosure.

The frame 40 is displayed in the first display region 35A, and the frame image 106A is displayed in a superimposed manner on the frame 40, as an example of the information based on the first region recognition result 106. In addition, as a part of the information included in the auxiliary information 44, the information based on the second region recognition result 110 and the information based on the feature recognition result 116 are displayed in the second display region 35B. A segmentation mask 110A is displayed in the second display region 35B, as an example of the information based on the second region recognition result 110. In addition, as examples of the information based on the feature recognition result 116, information indicating the type of the lesion 42 shown in the frame 40 displayed in the first display region 35A, information indicating the morphological type of the lesion 42, and information indicating the malignancy degree of the lesion 42 are displayed in the second display region 35B. Further, in the second display region 35B, as an example of the information based on the second region recognition result 110, information indicating the size of the lesion 42 shown in the frame 40 displayed in the first display region 35A is displayed. The size of the lesion 42 is calculated based on the number of pixels of the segmentation mask 110A.

The display of the information based on the first region recognition result 106, the display of the information based on the second region recognition result 110, and the display of the information based on the feature recognition result 116 are updated in synchronization with the display timing of each frame 40 included in the video image 39 displayed in the first display region 35A. Stated another way, the display of the information based on the first region recognition result 106, the display of the information based on the second region recognition result 110, and the display of the information based on the feature recognition result 116 are updated in accordance with the predetermined frame rate applied to the display of the frame 40.

In the following description, for convenience of description, in a case in which it is not necessary to distinguish between the information based on the first region recognition result 106, the information based on the second region recognition result 110, and the information based on the feature recognition result 116, the information based on the first region recognition result 106, the information based on the second region recognition result 110, and the information based on the feature recognition result 116 will be collectively referred to as “processing result information”.

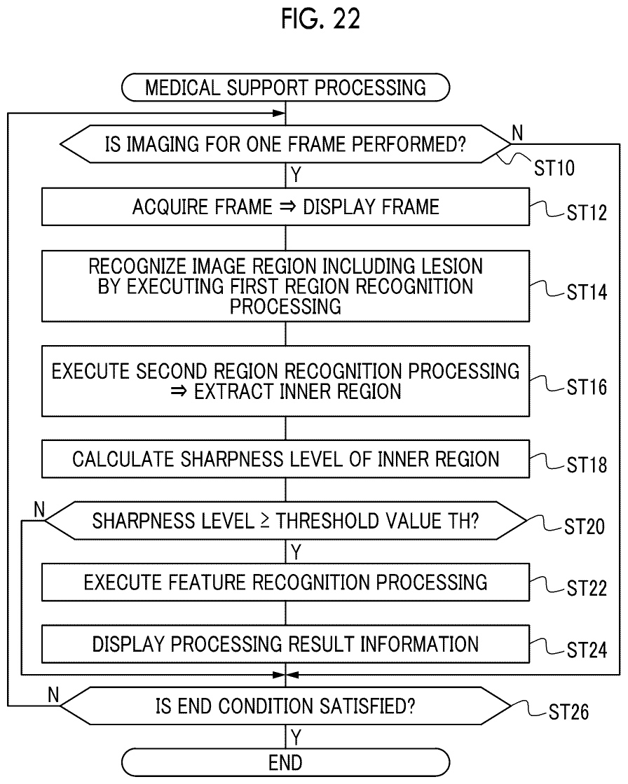

Hereinafter, an operation of a part of the endoscope system 10 according to the present disclosure will be described with reference to FIG. 22. The flowchart shown in FIG. 22 is an example of a “medical support method” according to the present disclosure.

In the medical support processing shown in FIG. 22, first, in step ST10, the recognition unit 82A determines whether or not imaging for one frame has been performed by the camera 52 in the large intestine 28. In step ST10, in a case in which the imaging for one frame is not performed by the camera 52 in the large intestine 28, a negative determination is made, and the medical support processing proceeds to step ST26. In step ST10, in a case in which the imaging for one frame has been performed by the camera 52 in the large intestine 28, an affirmative determination is made, and the medical support processing proceeds to step ST12.

In step ST12, the recognition unit 82A and the controller 82B acquire the frame 40 obtained by imaging the inside of the large intestine 28 (for example, the intestinal wall 32) with the camera 52 (see FIG. 8). The controller 82B displays the frame 40 in the first display region 35A (refer to FIG. 8). In a case in which the frame 40 is already displayed in the first display region 35A, the controller 82B updates the frame 40 displayed in the first display region 35A. Stated another way, by repeatedly executing the processing in step ST12, the frame 40 is displayed in the first display region 35A in a live view mode. After the processing of step ST12 is executed, the medical support processing proceeds to step ST14.

In step ST14, the recognition unit 82A recognizes the image region including the lesion 42 by executing the first region recognition processing 105 on the frame 40 acquired in step ST12, and generates the first region recognition result 106 that is the recognition result (see FIG. 8). After the processing of step ST14 is executed, the medical support processing proceeds to step ST16.

In step ST16, the recognition unit 82A extracts the processing target image 108 surrounded by the frame image 106A included in the first region recognition result 106 acquired in step ST14 from the frame 40 that is the execution target of the first region recognition processing 105, and executes the second region recognition processing 107 on the processing target image 108 (see FIG. 9). The recognition unit 82A executes the second region recognition processing 107 on the processing target image 108 to generate the second region recognition result 110 including the segmentation mask 110A (see FIG. 9). The recognition unit 82A executes processing of fitting the contour of the segmentation mask 110A within the lesion 42 on the processing target image 108 (see FIGS. 10, 14, and 18). The recognition unit 82A extracts, as the inner region 109, the image region surrounded by the processed contour of the segmentation mask 110A from the processing target image 108 (see FIGS. 11, 15, and 19). After the processing of step ST16 is executed, the medical support processing proceeds to step ST18.

In step ST18, the recognition unit 82A calculates the sharpness level 112 of the inner region 109 (see FIGS. 11, 15, and 19). After the processing in step ST18 is executed, the medical support processing proceeds to step ST20.

In step ST20, the recognition unit 82A determines whether or not the sharpness level 112 calculated in step ST18 is equal to or greater than the threshold value TH (see FIGS. 11, 15, and 19). In step ST20, in a case in which the sharpness level 112 is less than the threshold value TH, a negative determination is made, and the medical support processing proceeds to step ST26. In step ST20, in a case in which the sharpness level 112 is equal to or greater than the threshold value TH, an affirmative determination is made, and the medical support processing proceeds to step ST22.

In step ST22, the recognition unit 82A performs the feature recognition processing 114 on the inner region 109 (see FIGS. 12, 16, and 20). After the processing in step ST22 is executed, the medical support processing proceeds to step ST24.

In step ST24, the controller 82B acquires the first region recognition result 106, the second region recognition result 110, and the feature recognition result 116 from the recognition unit 82A, generates the processing result information, and displays the processing result information on the screen 35 (see FIG. 21). After the processing of step ST24 is executed, the medical support processing proceeds to step ST26.

In step ST26, the controller 82B determines whether or not a medical support processing end condition is satisfied. Examples of the medical support processing end condition include a condition that an instruction to end the medical support processing is issued to the endoscope system 10 (for example, a condition that the reception device 64 receives the instruction to end the medical support processing).

In a case in which the medical support processing end condition is not satisfied in step ST26, a negative determination is made, and the medical support processing proceeds to step ST10. In a case in which the medical support processing end condition is satisfied in step ST26, an affirmative determination is made, and the medical support processing ends.

As described above, for example, in a case in which the sharpness level of the processing target image 108 in which the lesion 42 is shown is calculated, the high-frequency component of the outer edge 42A of the lesion 42 affects the sharpness level, and thus there is a concern that excessively high sharpness level is calculated. In a case in which the excessively high sharpness level is calculated, it is determined that the processing target image 108 is an image suitable as the processing execution target of the feature recognition processing 114, and the feature recognition processing 114 is executed on the processing target image 108. However, in this case, there is a concern that the medical feature of the lesion 42 is erroneously recognized by the feature recognition processing 114 on the processing target image 108.

Therefore, in the present embodiment, the sharpness level 112 of the inner region 109, which is a region inside the outer edge 42A of the lesion 42 shown in the frame 40, is calculated. The inner region 109 is an image region extracted from the processing target image 108 by the contour of the segmentation mask 110A obtained by performing the processing of fitting the contour of the segmentation mask 110A within the lesion 42 on the processing target image 108. Therefore, since the sharpness level 112 of the inner region 109 is calculated as a value that is less likely to be affected by the high-frequency component of the outer edge 42A of the lesion 42, the sharpness level 112 is prevented from being an excessively high value due to the high-frequency component of the outer edge 42A of the lesion 42. In the present embodiment, the feature recognition processing 114 is performed or not performed on the inner region 109 in accordance with the sharpness level 112. Therefore, it is possible to prevent the information reflecting the result of the erroneous recognition of the medical feature of the lesion 42 by the feature recognition processing 114 due to the low accuracy of the sharpness level 112 from being provided to the doctor 12 or the like.

In addition, in the present embodiment, the recognition unit 82A performs, as the processing included in the first control processing 113, processing of executing the feature recognition processing 114 in a case in which the sharpness level 112 is equal to or greater than the threshold value TH and not executing the feature recognition processing 114 in a case in which the sharpness level 112 is less than the threshold value TH. In a case in which the sharpness level 112 is equal to or greater than the threshold value TH, the feature recognition processing 114 is executed on the inner region 109 that does not include the non-sharp image region, such as blurriness, in the processing target image 108. As a result, it is possible to provide the doctor 12 or the like with information reflecting the result of the medical feature of the lesion 42 is recognized with high accuracy by the feature recognition processing 114. On the other hand, in a case in which the sharpness level 112 is less than the threshold value TH, the feature recognition processing 114 is not executed on the inner region 109 affected by the non-sharp image region such as the blurriness, so that it is possible to prevent the information reflecting the result of the erroneous recognition of the medical feature of the lesion 42 by the feature recognition processing 114, from being provided to the doctor 12 or the like. In addition, it is found that the inner region 109 is affected by the non-sharp image region such as blurriness in a case in which the sharpness level 112 is less than the threshold value TH, and thus it is possible to prevent the unnecessary execution of the feature recognition processing 114.

Furthermore, in the present embodiment, the inner region 109 is generated based on the segmentation mask 110A obtained by executing the second region recognition processing 107 on the processing target image 108. Stated another way, an image region cut out from the processing target image 108 by an outer contour line obtained by offsetting the outer edge 110A1 of the segmentation mask 110A to the inside of the segmentation mask 110A is set as the inner region 109. Therefore, it is possible to easily obtain an image region having a small influence of the high-frequency component as the inner region 109 that is a calculation target of the sharpness level 112, as compared with a case in which the image region including the outer edge 42A of the lesion 42 shown in the processing target image 108 is the inner region 109. In addition, it is possible to easily obtain the image region with a high confidence level of the lesion 42 as the inner region 109, as compared with a case in which the image region including the outer edge 42A of the lesion 42 shown in the processing target image 108 is the inner region 109.

Further, in the present embodiment, the resolution of the segmentation mask 110A is lower than the resolution of the frame 40, and the processing of increasing the resolution of the segmentation mask 110A to the same resolution as the resolution of the frame 40 is not necessary for generating the inner region 109. Therefore, it is possible to reduce the processing load on the processor 82 as compared with a case in which the processing of increasing the resolution of the segmentation mask 110A to the same resolution as the resolution of the frame 40 is performed in order to generate the inner region 109.

In addition, in the present embodiment, in a case in which a part of the lesion 42 shown in the processing target image 108 deviates from the processing target image 108, an image region excluding the edge 40A of the frame 40 (in other words, an image region that does not include the edge 40A of the frame 40 among all the image regions of the processing target image 108) among all the image regions of the processing target image 108 is used as the inner region 109C. Accordingly, since the sharpness level 112C of the inner region 109C is calculated as a value that is less likely to be affected by the high-frequency component of the edge 40A of the frame 40, the sharpness level 112C is prevented from being an excessively high value due to the high-frequency component of the edge 40A of the frame 40. In the present embodiment, the feature recognition processing 114 is performed or not performed on the inner region 109C in accordance with the sharpness level 112C. Therefore, it is possible to prevent the information reflecting the result of the erroneous recognition of the medical feature of the lesion 42 by the feature recognition processing 114 due to the low accuracy of the sharpness level 112C from being provided to the doctor 12 or the like.

In addition, in the present embodiment, in a case in which the halation portion 117 is present in the lesion 42 shown in the processing target image 108, an image region outside the edge of the halation portion 117 shown in the processing target image 108 (that is, the edge of the halation portion 117 included as an image in the processing target image 108) among all image regions of the processing target image 108 is used as the inner region 109B. Accordingly, the sharpness level 112B of the inner region 109B is calculated as a value that is less likely to be affected by the high-frequency component of the edge of the halation portion 117, so that the sharpness level 112B is prevented from being a excessively high value due to the high-frequency component of the edge of the halation portion 117. In the present embodiment, the feature recognition processing 114 is performed or not performed on the inner region 109B in accordance with the sharpness level 112B. Therefore, it is possible to prevent the information reflecting the result of the erroneous recognition of the medical feature of the lesion 42 by the feature recognition processing 114 due to the low accuracy of the sharpness level 112B from being provided to the doctor 12 or the like.

In the embodiment described above, the form example has been described in which the image region surrounded by the outer edge 110A1 of the segmentation mask 110A having the reduced outer shape among all the image regions of the processing target image 108 is the inner region 109, but the present disclosure is not limited to this. For example, instead of the inner region 109, an image region within a second frame that is obtained by narrowing the first frame, which is the frame surrounding the lesion 42, to the inner side of the lesion 42 with respect to the outer edge 42A of the lesion 42 among all image regions of the processing target image 108 may be applied.

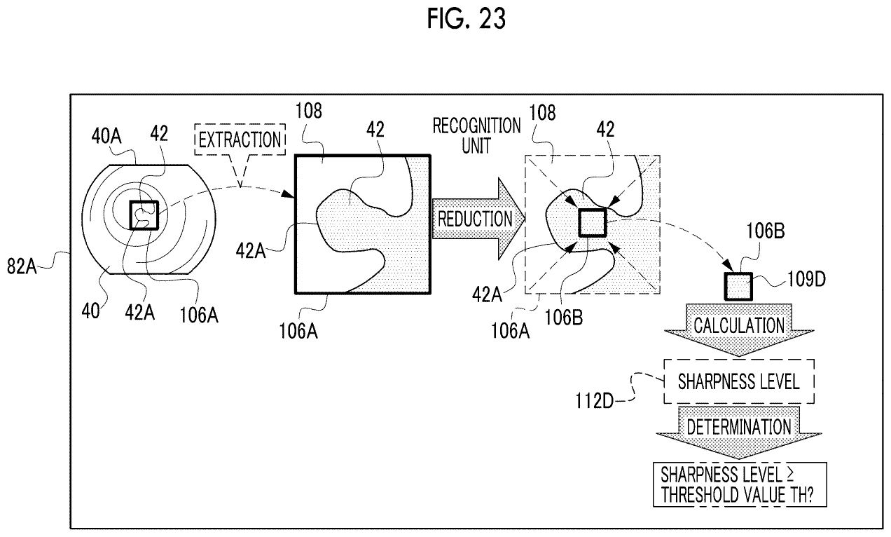

For example, as shown in FIG. 23, an image region surrounded by a frame image 106B obtained by reducing the frame image 106A to the inner side of the lesion 42 with respect to the outer edge 42A of the lesion 42 among all image regions of the processing target image 108 may be extracted by the recognition unit 82A, as an inner region 109D. In this case as well, the sharpness level 112D of the inner region 109D may be calculated in the same method as the sharpness level 112A to 112C. Examples of the frame image 106A include a frame image corresponding to the bounding box. In addition, here, although the form example has been described in which the frame image 106A is reduced to the inner side of the lesion 42 with respect to the outer edge 42A of the lesion 42, this is merely an example, and the frame image 106B may be determined by offsetting the outer edge of the frame image 106A to the inner side of the lesion 42 by the number of pixels determined in accordance with the geometrical characteristics of the lesion 42 and the geometrical characteristics of the frame image 106A.

In this way, even in a case in which the inner region 109D is extracted from the processing target image 108 by the frame image 106B obtained by narrowing the frame image 106A in the processing target image 108 to the inner side of the lesion 42 from the outer edge 42A of the lesion 42, the same effects as those of the embodiment described above can be obtained. In addition, in the example shown in FIG. 23, since the execution of the second region recognition processing 107 is not necessary, the processing load is reduced by the amount of the execution of the second region recognition processing 107 that is not necessary.

In addition, in the example shown in FIG. 23, the inner region 109D is an example of an “inner region” according to the present disclosure. Further, in the example shown in FIG. 23, the frame image 106A is an example of a “first frame” according to the present disclosure. Further, in the example shown in FIG. 23, the frame image 106B is an example of a “second frame” according to the present disclosure.

In the following description, for convenience of description, in a case in which it is not necessary to distinguish between the sharpness levels 112A, 112B, 112C, and 112D, the sharpness levels 112A, 112B, 112C, and 112D will be collectively referred to as a “sharpness level 112”. In addition, in the following description, for convenience of description, in a case in which it is not necessary to distinguish between the inner regions 109A, 109B, 109C, and 109D, the inner regions 109A, 109B, 109C, and 109D will be collectively referred to as an “inner region 109”.

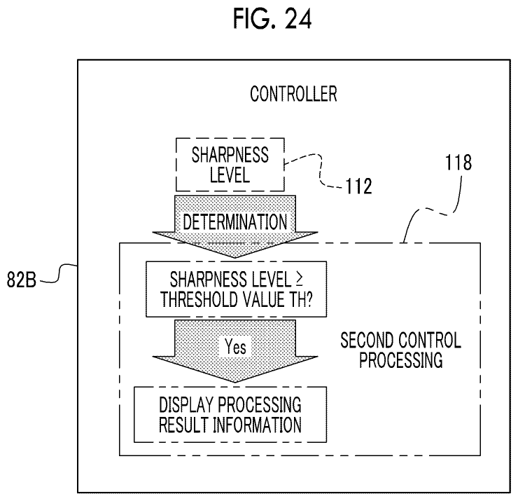

In the embodiment described above, although the form example has been described in which the first control processing 113 is executed by the recognition unit 82A, the present disclosure is not limited to this. For example, as shown in FIG. 24, second control processing 118 executed by the controller 82B may be applied instead of the first control processing 113 executed by the recognition unit 82A. The second control processing 118 is an example of “second processing” according to the present disclosure.

In the example shown in FIG. 12, the controller 82B executes the second control processing 118. The second control processing 118 is processing of controlling the output of the processing result information (for example, processing of strengthening or weakening the output level of the processing result information) in accordance with the sharpness level 112. As the processing included in the second control processing 118, the controller 82B performs processing of outputting the processing result information in a case in which the sharpness level 112 is equal to or greater than the threshold value TH and not outputting the processing result information in a case in which the sharpness level 112 is less than the threshold value TH.

For example, the output of the processing result information is implemented by displaying the processing result information on the screen 35. Stated another way, in a case in which the sharpness level 112 is equal to or greater than the threshold value TH, the processing result information is displayed on the screen 35, and in a case in which the sharpness level 112 is less than the threshold value TH, the processing result information is not displayed on the screen 35. Here, the concept of “the processing result information is not displayed on the screen 35” also includes a meaning that the display intensity of the processing result information is weakened as compared with the display intensity of the processing result information in a case in which the sharpness level 112 is equal to or greater than the threshold value TH (for example, a meaning that the processing result information is displayed with a transparency that is visually imperceptible), and a meaning that the processing result information is displayed in a state of being visually imperceptible by being masked.

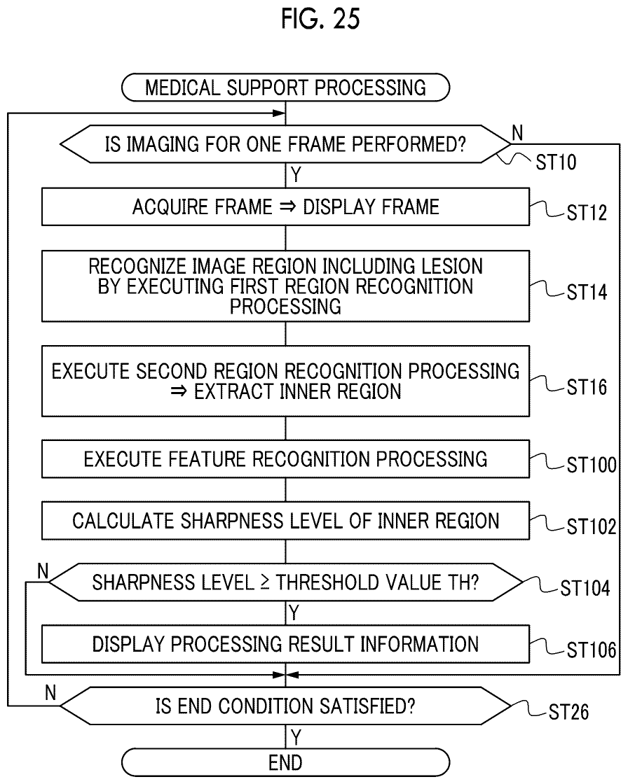

FIG. 25 shows an example of a flow of the medical support processing in a case in which the second control processing 118 is executed by the controller 82B. The flowchart shown in FIG. 25 is different from the flowchart shown in FIG. 22 in that each processing of step ST100 to step ST106 is provided instead of each processing of step ST18 to step ST24.

In step ST100, the recognition unit 82A executes the feature recognition processing 114 on the inner region 109 in the same method as in the embodiment described above. After the processing in step ST100 is executed, the medical support processing proceeds to step ST102.

In step ST102, the recognition unit 82A calculates the sharpness level 112 of the inner region 109 in the same method as in the embodiment described above. After the processing in step ST102 is executed, the medical support processing proceeds to step ST104.

In step ST104, the controller 82B determines whether or not the sharpness level 112 is equal to or greater than the threshold value TH. In step ST104, in a case in which the sharpness level 112 is less than the threshold value TH, a negative determination is made, and the medical support processing proceeds to step ST26. In step ST104, in a case in which the sharpness level 112 is equal to or greater than the threshold value TH, an affirmative determination is made, and the medical support processing proceeds to step ST106.

In step ST106, the controller 82B acquires the first region recognition result 106, the second region recognition result 110, and the feature recognition result 116 from the recognition unit 82A in the same method as in the embodiment described above, generates processing result information, and displays the processing result information on the screen 35. After the processing in step ST106 is executed, the medical support processing proceeds to step ST26.

As described above, in the examples shown in FIGS. 24 and 25, the feature recognition processing 114 is executed on the inner region 109 regardless of the sharpness level 112. The difference from the embodiment described above is that the processing result information is displayed or not displayed on the screen 35 in accordance with the sharpness level 112. As described above, since the processing result information is displayed or not displayed on the screen 35 in accordance with the sharpness level 112, it is possible to prevent the information reflecting the result of the erroneous recognition of the medical feature of the lesion 42 by the feature recognition processing 114 due to the low certainty of the sharpness level 112 from being provided to the doctor 12 or the like.

In addition, in the examples shown in FIGS. 24 and 25, in a case in which the sharpness level 112 is equal to or greater than the threshold value TH, the processing result information obtained by executing the feature recognition processing 114 on the inner region 109 that does not include the non-sharp image region or the like in the processing target image 108 is displayed on the screen 35. As a result, it is possible to provide the doctor 12 or the like with information reflecting the result of the medical feature of the lesion 42 is recognized with high accuracy by the feature recognition processing 114. On the other hand, in a case in which the sharpness level 112 is less than the threshold value TH, the processing result information obtained by executing the feature recognition processing 114 on the inner region 109 affected by the unclear image region such as the blurriness is not displayed on the screen 35, so that it is possible to prevent the information reflecting the result of the medical feature of the lesion 42 that is erroneously recognized by the feature recognition processing 114 from being provided to the doctor 12 or the like. In addition, in a case in which the sharpness level 112 is less than the threshold value TH, it is found that the inner region 109 is affected by the non-sharp image region such as blurriness, so that it is possible to prevent the unnecessary output of the processing result information (for example, display on the screen 35).

In the embodiment described above, the form example has been described in which the first control processing 113 is executed in accordance with the sharpness level 112 obtained from the single processing target image 108, but this is merely an example. For example, the first control processing 113 may be performed in accordance with a plurality of sharpness levels 112 obtained from a plurality of processing target images 108 corresponding to the plurality of frames 40 arranged in time series. Here, a specific form example thereof will be described.

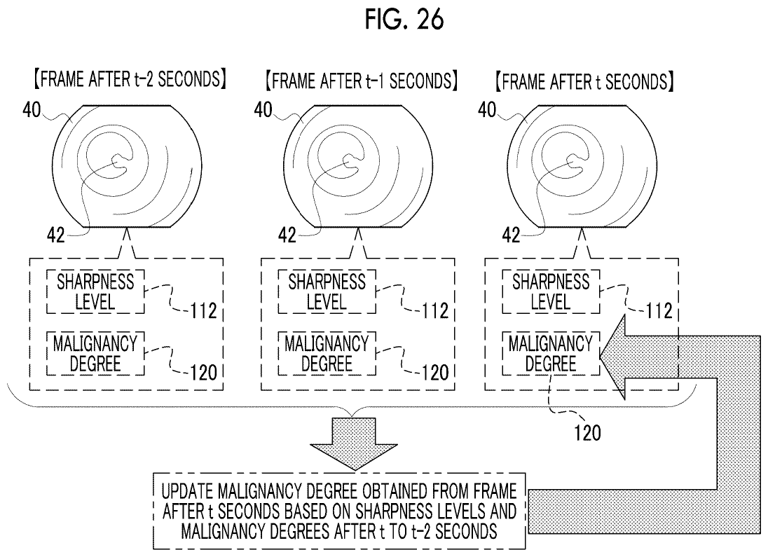

The feature recognition processing 114 included in the first control processing 113 is executed on each of a plurality of inner regions 109 obtained from the plurality of processing target images 108 (that is, three processing target images 108) corresponding to the plurality of frames 40 (that is, three frames 40) obtained after t seconds, after t−1 seconds, and after t−2 seconds. As a result, as shown in FIG. 26 as an example, a plurality of malignancy degrees 120 are obtained. The malignancy degree 120 is information included in the feature recognition result 116.

A weight determined based on the plurality of sharpness levels 112 corresponding to the plurality of processing target images 108 is given to a latest feature recognition result 116 (that is, the feature recognition result 116 obtained by executing the feature recognition processing 114 on the processing target image 108 corresponding to the frame 40 after t seconds) among a plurality of feature recognition results 116 (that is, three feature recognition results 116) obtained after t seconds, after t−1 seconds, and after t−2 seconds. For example, as shown in FIG. 26, the weight to be given to the malignancy degree 120 after t seconds is determined based on three sharpness levels 112 obtained after t seconds, after t−1 seconds, and after t−2 seconds and three malignancy degrees 120 included in the three feature recognition results 116, and the malignancy degree 120 after t seconds is updated by giving the weight to the malignancy degree 120 after t seconds.

For example, in a case in which three sharpness levels 112 and three malignancy degrees 120 of after t seconds, after t−1 seconds, and after t−2 seconds are obtained as shown in Table 1, the malignancy degree 120 of after t seconds is calculated as “0.756” by the following mathematical expression (1).

TABLE 1

| | After t − | After t − | |
|---|---|---|---|
| | 2 seconds | 1 seconds | After t seconds |
| Sharpness level | 10 | 100 | 15 |
| Malignancy degree | 0.4 | 0.8 | 0.7 |

$$Malignancy degree after t seconds = \frac{10}{( 10 + 100 + 15 )} \times 0.4 + \frac{100}{( 10 + 100 + 15 )} \times 0.8 + \frac{15}{( 10 + 100 + 15 )} \times 0.7 \qquad ( 1 )$$

Stated another way, the malignancy degree 120 included in the feature recognition result 116 in a case in which the feature recognition processing 114 is executed on the inner region 109 obtained from the processing target image 108 corresponding to the frame 40 obtained after t seconds is “0.7”, but the malignancy degree 120 after t seconds is updated from “0.7” to “0.756” by giving the weight of “1.08” to “0.7”. The malignancy degree 120 calculated by Expression (1) may be classified into any one of a grade 1, a grade 2, or a grade 3 and output to the screen 35 or the like.

In this way, it is possible to increase the contribution to the feature recognition result 116 (here, as an example, the malignancy degree 120) by the inner region 109 having a high sharpness level 112 among the three sharpness levels 112 of after t seconds, after t−1 seconds, and after t−2 seconds, and to decrease the contribution to the feature recognition result 116 (here, as an example, the malignancy degree 120) by the inner region 109 having a low sharpness level 112 among the three sharpness levels 112 of after t seconds, after t−1 seconds, and after t−2 seconds. As a result, a reliability degree of the latest feature recognition result 116 (here, as an example, a reliability degree of the malignancy degree 120 included in the feature recognition result 116 after t seconds) can be increased.

Here, although the form example has been described in which the weight is given to the malignancy degree 120 included in the feature recognition result 116 after t seconds, this is merely an example, and the weight may be given to the malignancy degree 120 included in each of the plurality of feature recognition results 116.

In addition, here, although the form example has been described in which the first control processing 113 is performed in accordance with the plurality of sharpness levels 112 obtained from the plurality of processing target images 108 corresponding to the plurality of frames 40 arranged in time series, this is merely an example. For example, the second control processing 118 may be performed in accordance with a plurality of sharpness levels 112 obtained from a plurality of processing target images 108 corresponding to the plurality of frames 40 arranged in time series. In such a case, since the reliability degree of the feature recognition result 116 corresponds to the sharpness level 112 (that is, the higher the sharpness level 112, the higher the reliability degree of the feature recognition result 116), the information based on the feature recognition result 116 may be displayed or not displayed in accordance with the reliability degree of the feature recognition result 116. For example, in a case in which the reliability degree of the feature recognition result 116 is equal to or higher than a predetermined level, the information based on the feature recognition result 116 may be displayed, and in a case in which the reliability degree of the feature recognition result 116 is lower than the predetermined level, the information based on the feature recognition result 116 may not be displayed.

In the embodiment described above, the threshold value TH has been described as an example, but a first threshold value and a second threshold value that is a value less than the first threshold value may be used instead of the threshold value TH. Examples of the first threshold value include the same value as the threshold value TH. In a case in which the first threshold value and the second threshold value that is a value less than the first threshold value are used, it is determined whether or not the sharpness level 112 is equal to or greater than the first threshold value, and in a case in which the sharpness level 112 is equal to or greater than the first threshold value, processing after step ST22 shown in FIG. 22 is executed or processing after step ST106 shown in FIG. 25 is executed.

On the other hand, in a case in which the sharpness level 112 is less than the first threshold value and less than the second threshold value, a negative determination is made in step ST20 shown in FIG. 22 and the processing of step ST26 is executed, or a negative determination is made in step ST104 shown in FIG. 25 and the processing of step ST26 is executed. In addition, in a case in which the sharpness level 112 is equal to or greater than the second threshold value and less than the first threshold value, the doctor 12 or the like may be allowed to decide whether or not to cause the recognition unit 82A to execute the feature recognition processing 114 or to cause the controller 82B to output the processing result information.

In addition, in a case in which the sharpness level 112 is equal to or greater than the second threshold value and less than the first threshold value, the recognition unit 82A may be caused to execute the feature recognition processing 114 or the controller 82B may be caused to output the processing result information, in accordance with a predetermined condition. For example, in a case in which the sharpness level 112 is equal to or greater than the second threshold value and less than the first threshold value, the recognition unit 82A may be caused to execute the feature recognition processing 114 on a condition that a portion shown in the frame 40 (that is, a portion included in the frame 40 as an image) displayed in the first display region 35A is an important portion. In addition, in a case in which the sharpness level 112 is equal to or greater than the second threshold value and less than the first threshold value, the processing result information may be output to the controller 82B on a condition that the portion shown in the frame 40 displayed in the first display region 35A is an important portion.

In the embodiment described above, the calculation method in which the processing of filtering the inner region 109 using the Laplacian filter is incorporated has been described as the method for calculating the sharpness level 112, but this is merely an example, and a calculation method in which processing of filtering the inner region 109 using a Sobel filter is incorporated may be used. In addition, a calculation method may be adopted in which the inner region 109 is converted into a frequency region by performing a fast Fourier transform on the inner region 109, a high-frequency component of the frequency region is enhanced by applying a high-pass filter to the frequency region, and the frequency region is converted into a spatial region by performing an inverse fast Fourier transform on the frequency region in which the high-frequency component is enhanced.

In the embodiment described above, as the method of calculating the sharpness level 112, the method of calculating the sharpness level 112 by averaging the total value of the absolute values of the pixel values of each pixel of the inner region 109 to which the Laplacian filter is applied has been described, but this is merely an example. Instead of the method of averaging the total value of the absolute values of the pixel values of the respective pixels of the inner region 109 to which the Laplacian filter is applied, a statistic other than the average value may be calculated as the sharpness level 112. Examples of the statistic other than the average value include a variance, a standard deviation, a median value, a maximum value, and a minimum value. In addition, the statistic may be a combination of two or more of an average value, a variance, a standard deviation, a median value, a maximum value, or a minimum value.

In the embodiment described above, the endoscopic image has been described as the frame 40, but this is merely an example, and the present disclosure is established even in a case in which a medical image, such as an MRI image, a CT image, or an X-ray image, is applied instead of the frame 40.

In the embodiment described above, the first region recognition processing 105 using AI of the bounding box method has been described as an example, but this is merely an example, and for example, object recognition processing using AI of the segmentation method may be performed instead of the first region recognition processing 105 using AI of the bounding box method. In addition, instead of the AI-based recognition processing, recognition processing using a non-AI-based method (for example, the template matching method) may be executed, or recognition processing using a combination of the non-AI-based method and the AI-based method may be executed.

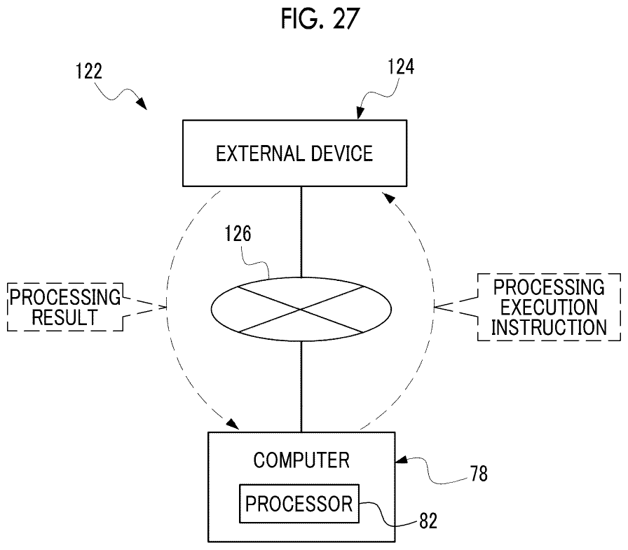

In the embodiment described above, the form example has been described in which the medical support processing is executed by the computer 78, but the present disclosure is not limited to this, and at least a part of the medical support processing may be executed by a device provided outside the computer 78. Hereinafter, an example of this case will be described with reference to FIG. 27.

FIG. 27 is a conceptual diagram showing an example of a configuration of an endoscope system 122. The endoscope system 122 is an example of an “endoscope system” according to the present disclosure. The endoscope system 122 is different from the endoscope system 10 according to the embodiment described above in that an external device 124 is provided.

The external device 124 is connected communicably to the computer 78 via a network 126 (for example, a WAN and/or a LAN).

Examples of the external device 124 include at least one server that directly or indirectly transmits and receives data to and from the computer 78 via the network 126. The external device 124 receives a processing execution instruction issued from the processor 82 of the computer 78 via the network 126. Then, the external device 124 executes processing corresponding to the received processing execution instruction, and transmits a processing result to the computer 78 via the network 126. In the computer 78, the processor 82 receives the processing result transmitted from the external device 124 via the network 126, and executes processing using the received processing result.

Examples of the processing execution instruction include an instruction for the external device 124 to execute at least a part of the medical support processing. A first example of at least a part of the medical support processing (that is, processing to be executed by the external device 124) is the first region recognition processing 105. In such a case, the external device 124 executes the first region recognition processing 105 in response to the processing execution instruction issued from the processor 82 via the network 126, and transmits the first region recognition result 106 to the computer 78 via the network 126. In the computer 78, the processor 82 receives the first region recognition result 106, and executes the same processing as in the embodiment described above using the received first region recognition result 106.

A second example of at least a part of the medical support processing (that is, processing to be executed by the external device 124) is the second region recognition processing 107. In such a case, the external device 124 executes the second region recognition processing 107 in response to the processing execution instruction issued from the processor 82 via the network 126, and transmits the second region recognition result 110 to the computer 78 via the network 126. In the computer 78, the processor 82 receives the second region recognition result 110, and executes the same processing as in the embodiment described above using the received second region recognition result 110.

A third example of at least a part of the medical support processing (that is, the processing to be executed by the external device 124) is the processing performed by the first control processing 113. In such a case, the external device 124 executes the first control processing 113 in response to the processing execution instruction issued from the processor 82 via the network 126, and transmits the processing result (for example, information indicating whether or not the feature recognition processing 114 has been executed, and/or the feature recognition result 116 obtained in a case in which the feature recognition processing 114 has been executed) to the computer 78 via the network 126. In the computer 78, the processor 82 receives the processing result and executes the same processing (for example, the display using the display device 18) as the processing in the embodiment described above using the received processing result.

A fourth example of at least a part of the medical support processing (that is, the processing to be executed on the external device 124) is the extraction of the processing target image 108 from the frame 40, the extraction of the inner region 109 from the processing target image 108, and/or the calculation of the sharpness level 112. In such a case, the external device 124 executes the extraction of the processing target image 108 from the frame 40, the extraction of the inner region 109 from the processing target image 108, and/or the calculation of the sharpness level 112 in accordance with the processing execution instruction issued from the processor 82 via the network 126, and transmits the processing result (for example, the processing target image 108, the inner region 109, and/or the sharpness level 112) to the computer 78 via the network 126. In the computer 78, the processor 82 receives the processing result and executes the same processing as the processing in the embodiment described above using the received processing result.

The external device 124 may be implemented by cloud computing. The cloud computing is merely an example, and the external device 124 may be implemented by network computing, such as fog computing, edge computing, or grid computing.

In the embodiment described above, the form example has been described in which the medical support program 98 is stored in the storage 86, but the present disclosure is not limited to this. For example, the medical support program 98 may be stored in a portable non-transitory computer-readable storage medium such as an SSD or a USB memory. The medical support program 98, which is stored in the non-transitory storage medium, is installed in the computer 78 of the endoscope system 10. The processor 82 executes the medical support processing in accordance with the medical support program 98.

Further, the medical support program 98 may be stored in a storage device of another computer, a server, or the like that is connected to the endoscope system 10 via the network, and the medical support program 98 may be downloaded and installed in the computer 78 in response to a request from the endoscope system 10.

It is not necessary to store the entire medical support program 98 in a storage device of another computer or a server device connected to the endoscope system 10 or to store the entire medical support program 98 in the storage 86, and a part of the medical support program 98 may be stored.

The following various processors can be used as hardware resources for executing the medical support processing. An example of the processor is a CPU that is a general-purpose processor that executes software, that is, a program, to function as the hardware resource for executing the medical support processing. Another example of the processor is a dedicated electric circuit that is a processor having a dedicated circuit configuration designed to execute specific processing, such as an FPGA, a PLD, or an ASIC. All processors have a memory built therein or connected thereto, and all processors use the memory to execute the medical support processing.

The hardware resource for executing the medical support processing may be configured by one of the various processors or by a combination of two or more processors of the same type or different types (for example, a combination of a plurality of FPGAs or a combination of a CPU and an FPGA). Furthermore, the hardware resource for executing the medical support processing may be one processor.

A first example of the configuration in which the hardware resource is configured by one processor is an aspect in which one processor is configured by a combination of one or more CPUs and software, and this processor functions as the hardware resource for executing the medical support processing. As a second example, as typified by an SoC or the like, there is a form in which a processor that implements all functions of a system including a plurality of hardware resources executing the medical support processing with one IC chip is used. In this way, the medical support processing is implemented by using one or more of the various processors as the hardware resource.

Furthermore, as the hardware structure of the various processors, specifically, an electronic circuit in which circuit elements, such as semiconductor elements, are combined can be used. The above-described medical support processing is merely an example. Therefore, it goes without saying that unnecessary steps may be deleted, new steps may be added, or the processing order may be changed, within a range that does not deviate from the gist of the present disclosure.

The above-described contents and the above-shown contents are the detailed description of the parts according to the present disclosure, and are merely examples of the present disclosure. For example, the descriptions of the configurations, the functions, the operations, and the effects are the descriptions of the examples of the configurations, the functions, the operations, and the effects of the parts according to the present disclosure. Therefore, it goes without saying that unnecessary parts may be deleted, new elements may be added, or replacements may be made with respect to the above-described contents and the above-shown contents within a range that does not deviate from the gist of the present disclosure. In order to avoid confusion and to facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which each of the documents, the patent applications, and the technical standards are specifically and individually stated to be described by reference.

What is claimed is:

1. A medical support device comprising:
a processor configured to:
acquire a medical image obtained by imaging a portion including a feature region; and
perform first processing in accordance with a sharpness level of an inner region, which is a region inside an outer edge of the feature region included in the medical image, or second processing in accordance with the sharpness level,
wherein the first processing is processing of controlling image recognition processing that is executable on the medical image and that recognizes a feature of the feature region, and
the second processing is processing of controlling output of information based on a processing result of the image recognition processing.

2. The medical support device according to claim 1,
wherein the first processing includes processing of executing the image recognition processing in a case in which the sharpness level is equal to or greater than a first threshold value and not executing the image recognition processing in a case in which the sharpness level is less than a second threshold value that is equal to or less than the first threshold value.

3. The medical support device according to claim 1,
wherein the second processing includes processing of outputting the information in a case in which the sharpness level is equal to or greater than a first threshold value and not outputting the information in a case in which the sharpness level is less than a second threshold value that is equal to or less than the first threshold value.

4. The medical support device according to claim 1,
wherein region recognition processing of recognizing the feature region is executed on the medical image, and
the inner region is a region based on a segmentation mask obtained by executing the region recognition processing.

5. The medical support device according to claim 4,
wherein a resolution of the segmentation mask is lower than a resolution of the medical image.

6. The medical support device according to claim 1,
wherein the inner region is a region within a second frame that is a frame obtained by narrowing a first frame, which is a frame surrounding the feature region, to an inner side of the feature region with respect to the outer edge.

7. The medical support device according to claim 6,
wherein region recognition processing of recognizing the feature region is executed on the medical image,
a bounding box is used in the region recognition processing, and
the first frame corresponds to the bounding box.

8. The medical support device according to claim 1,
wherein, in a case in which a part of the feature region included in the medical image deviates from the medical image, the inner region is a region excluding an edge of the medical image.

9. The medical support device according to claim 1,
wherein, in a case in which a halation portion exists in the feature region included in the medical image, the inner region is a region outside an edge of the halation portion.

10. The medical support device according to claim 1,
wherein the first processing and/or the second processing is executed in accordance with a plurality of the sharpness levels obtained from a plurality of the medical images arranged in time series.

11. The medical support device according to claim 1,
wherein a weight determined based on a plurality of the sharpness levels is given to at least one processing result among a plurality of the processing results obtained by executing the image recognition processing on the plurality of medical images.

12. The medical support device according to claim 1,
wherein the output of the information includes display of the information on a screen.

13. The medical support device according to claim 1,
wherein the feature is a medical feature of the feature region.

14. The medical support device according to claim 1,
wherein the feature region is a lesion.

15. The medical support device according to claim 1,
wherein the medical image is an endoscopic image.

16. An endoscope system comprising:
the medical support device according to claim 1; and
an endoscope in which an image sensor that images the portion is mounted.

17. A medical support method comprising:
acquiring a medical image obtained by imaging a portion including a feature region; and
performing first processing in accordance with a sharpness level of an inner region, which is a region inside an outer edge of the feature region included in the medical image, or second processing in accordance with the sharpness level,
wherein the first processing is processing of controlling image recognition processing that is executable on the medical image and that recognizes a feature of the feature region, and
the second processing is processing of controlling output of information based on a processing result of the image recognition processing.

18. A non-transitory computer-readable storage medium storing a program executable by a computer to execute medical support processing comprising:
acquiring a medical image obtained by imaging a portion including a feature region; and
performing first processing in accordance with a sharpness level of an inner region, which is a region inside an outer edge of the feature region included in the medical image, or second processing in accordance with the sharpness level,
wherein the first processing is processing of controlling image recognition processing that is executable on the medical image and that recognizes a feature of the feature region, and
the second processing is processing of controlling output of information based on a processing result of the image recognition processing.
